(19) United States
(12) Patent Application Publication
(10) Pub. No.: US 2013/0324866 A1
(43) Pub. Date: Dec. 5, 2013

(54) INDICATIONS OF CROSS-SECTION OF SMALL BRANCHED BLOOD VESSELS

(71) Applicant: Reuven Gladshtein, Netanya (IL)

(72) Inventor: Reuven Gladshtein, Netanya (IL)

(73) Assignee: VITA-SENTRY LTD., Nentanya (IL)

(21) Appl. No.: 13/985,068

(22) PCT Filed: Feb. 14, 2012
(86) PCT No.: PCT/IB2012/050672

§ 371 Date: Aug. 13, 2013

Related U.S. Application Data
(60) Provisional application No. 61/463,049, filed on Feb. 14, 2011.
(60) Provisional application No. 61/631,923, filed on Jan. 17, 2012.

(51) Int. Cl. A61B 5/02; A61B 5/0295; A61B 5/026

(52) U.S. Cl. CPC A61B 5/02007; A61B 5/0261; A61B 5/0295
USPC 600/507; 600/504

(57) ABSTRACT

Systems and methods of extracting information relating to diameter and/or diameter changes in small blood vessels such as arterioles. This information may be used to assess a degree of vasoconstriction and/or vasodilatation. In one method, changes in vessel cross-section due to pulse wave arrival is assessed in both arterioles and in larger arteries. A time delay between the changes and/or a change in time delay is optionally associated with arteriole cross-section and/or changes therein.

### RELATED APPLICATION/S

This application claims the benefit of priority under 35 USC 119(e) of U.S. Provisional Patent Application No. 61/463,049 filed Feb. 14, 2011, and of a U.S. Provisional Patent Application titled “Method and apparatus for monitoring cross section size variations in branched blood vessels,” listing Reuven Gladshtein as the inventor, and filed about Jan. 17, 2012, the contents of which are incorporated herein by reference in their entirety.

### FIELD AND BACKGROUND OF THE INVENTION

The present invention, in some embodiments thereof, relates to a method and apparatus for measuring vascular parameters and, more particularly, but not exclusively, to a method and apparatus for monitoring changes in the cross section of small branching arteries and arterioles.

Many medical conditions are characterized by changes or abnormalities in the diameter and cross sectional size and shape of small blood vessels. Vasoconstriction and vasodilation are reversible changes in the diameters of small blood vessels, in particular the arterioles. Vasoconstriction and vasodilation also play a role in regulating blood pressure, and in diseases characterized by abnormal regulation of blood pressure (hypertension and hypotension). Other diseases are characterized by irreversible changes in the diameters and cross sections of small blood vessels, including diabetes and atherosclerosis.

Generally, arterioles are too small to image, using such imaging methods as ultrasound, MRI, and x-rays, including CT scans.

Other techniques for examining the circulatory system are known, for example. sphyngomanometry provides data on systolic and diastolic blood pressure, and pulse oximetry provides data on blood oxygen levels. Arterial line and central venous line sensors provide data on blood pressure and blood flow rate inside large blood vessels.

Josep Sola, Stefano F. Rimoldi, and Yves Allemann, “Ambulatory Monitoring of the Cardiovascular System: the role of Pulse Wave Velocity,” in New Developments in Biomedical Engineering, Chapter 21, p. 391-422, provides a review of techniques for measuring pulse wave velocity, primarily in large arteries over large distances, for example from the heart to the extremities.

WO2007/097702 discusses a method for the generation, detection and evaluation of a photoplethysmographic (PPG) signal to monitor blood characteristics, in which the light source(s) are spaced at particular distances from photodetector(s). U.S. Pat. No. 6,123,719, U.S. Pat. No. 5,891,022, US2009/0306487 and EP1297784 discuss photoplethysmographic measurement systems that have at least two light emitters, each emitting light at different wavelengths and a photodiode for detecting the intensity of light reflected from a patient's tissue such as blood, finger, etc.

Additional background art includes Minnan Xu, “Local Measurement of the Pulse Wave Velocity Using Doppler Ultrasound,” M.S. thesis, Dept. of Electrical Engineering and Computer Science, M.I.T., May 24, 2002; A. C. Fowler and M. J. McGuinness, “A Delay Recruitment Model of the Cardiovascular Control System,” submitted to Journal of Mathematical Biology, June 2004, revised December 2004; John Allen, “Photoplethysmography and its application in clinical physiological measurement,” Physiol. Meas. 28 (2007), R1-R39; H. S. Lim and G. Y. H. Lip, “Arterial stiffness in diabetes and hypertension,” Journal of Human Hypertension (2004) 18, 467-468; and Emilie Franceschini, Bruno Lombard, and Joel Piraux, “Ultrasound characterization of red blood cells distribution: a wave scattering simulation study,” Journal of Physics: Conference Series 269 (2011) 012014.

### SUMMARY OF THE INVENTION

An aspect of some embodiments of the invention concerns finding a measure of a pressure wave in a blood vessel, and in smaller blood vessels that branch off it, and using differences between them to find information about the cross section, and/or about changes in the cross section, of the smaller blood vessels.

There is provided in accordance with an exemplary embodiment of the invention, a system for measuring an indication of blood vessel cross section in a subject, the system comprising:
a) a sensor adapted to perform measurements indicative of a pressure wave in blood vessels in the subject, and to generate first and second signals of the measurements, wherein a larger blood vessel contributes more, relative to smaller blood vessels branching off it, for the first signal than for the second signal; and
b) a signal processor adapted to find a time delay between the first and second signals, and to use the time delay to find the indication of cross section for the smaller blood vessels.

In an exemplary embodiment of the invention, the sensor is adapted to perform the measurements when it is placed adjacent to a surface of the subject's body, with the pressure wave contributing to the first signal from deeper beneath the surface, on average, than the pressure wave contributes to the second signal. Optionally or alternatively, the signal processor is adapted to find the time delay by finding a difference in timing in a minimum of the pressure wave for the first and second signals. Optionally or alternatively, the signal processor is adapted to find the time delay by finding a difference in timing in a maximum rate of increase of the pressure wave for the first and second signals. Optionally or alternatively, the signal processor is adapted to find the time delay by finding a difference in timing in a maximum of the pressure wave for the first and second signals.

In an exemplary embodiment of the invention, the system is adapted to perform the measurements non-invasively. Optionally or alternatively, the system is adapted to perform the measurements inside the body, or when a internal part of the body is exposed during surgery, for example, being mounted on a catheter, endoscope or other intrabody probe.

In an exemplary embodiment of the invention, the measurements indicative of a pressure wave comprise measurements of blood volume in the larger blood vessel and in the smaller blood vessels branching off from it. Optionally or alternatively, the measurements indicative of a pressure wave comprise measurements of flow rate in the larger blood vessel and in the smaller blood vessels branching off from it. Optionally or alternatively, the measurements indicative of a pressure wave comprise optical measurements. Optionally, the sensor is adapted to perform the measurements when it is placed adjacent to a surface of the subject's body, and the first signal is a signal of an optical measurement using a first set of wavelengths of light, while the second signal is a signal of an optical measurement using a second set of wavelengths of light that do not penetrate as far beneath the surface of the body as the first set of wavelengths of light.

In an exemplary embodiment of the invention, the sensor comprises:
a) a first light source and first detector adapted to be placed a first distance apart on a surface of the subject's body, light from the first light source scattering from beneath the surface to the first detector to generate the first signal; and
b) a second light source and a second light detector, one or both of them different respectively from the first light source and the first light detector, adapted to be placed a second distance apart, smaller than the first distance, light from the second light source scattering from beneath the surface to the second detector to generate the second signal.

Optionally or alternatively, the measurements comprise one or more of measurements of oxygen level and measurements of carbon dioxide level, in the blood or tissue or both.

In an exemplary embodiment of the invention, the measurements indicative of a pressure wave comprise ultrasound measurements. Optionally, the sensor is adapted to perform the measurements when it is placed adjacent to a surface of the subject's body, and the first signal is a signal of an ultrasound measurement using a first set of frequencies of ultrasound, while the second signal is a signal of an ultrasound measurement using a second set of frequencies of ultrasound that do not penetrate as far beneath the surface of the body as the first set of wavelengths of light.

In an exemplary embodiment of the invention, the sensor comprises:
a) a first ultrasound transducer and first receiver adapted to be placed a first distance apart on a surface of the subject's body, ultrasound from the first ultrasound transducer scattering from beneath the surface to the first receiver to generate the first signal; and
b) a second ultrasound transducer, the same as or different from the first ultrasound transducer, and a second receiver, the same as or different from the first receiver, adapted to be placed a second distance apart on the surface of the subject's body, smaller than the first distance, ultrasound from the second ultrasound transducer scattering from beneath the surface to the second receiver to generate the second signal.

In an exemplary embodiment of the invention, the measurements indicative of a pressure wave comprise electrical impedance measurements. Optionally, the sensor comprises:
a) a first pair of electrode units, adapted to be placed on a surface of the subject's body a first distance apart, and to measure a first impedance between them to generate the first signal; and
b) a second pair of electrode units, one or both of the units in the second pair differing from the electrode units in the first pair, adapted to be placed on a surface of the subject's body a second distance apart, shorter than the first distance, and to measure a second impedance between them to generate the second signal.

In an exemplary embodiment of the invention, the sensor has a time resolution better than 20 milliseconds.

In an exemplary embodiment of the invention, the signal processor is adapted to use the time delay by comparing the time delay to a threshold value for that subject, stored in a memory of the signal processor, and to find the indication that the smaller blood vessels are undergoing vasoconstriction if the time delay is greater than the threshold value.

There is provided in accordance with an exemplary embodiment of the invention a system for measuring an indication of blood vessel cross section in a subject, the system comprising:
a) a sensor adapted to perform at least a first set of measurements indicative of a pressure wave in blood vessels in the subject, and to generate at least a first signal of the first set of measurements; and
b) a signal processor adapted to find a first quantity indicative of rise time or rise rate of the pressure wave from the first signal, and to use the first quantity to find the indication of cross section for the blood vessels. Optionally, the sensor is adapted to perform a second set of measurements indicative of a pressure wave in blood vessels in the subject and to generate a second signal, wherein a larger blood vessel contributes more, relative to smaller blood vessels branching off it, for the first signal than for the second signal, and wherein the signal processor is adapted to find a second quantity indicative of rise time or rise rate of the pressure wave from the second signal, and to use a comparison of the first quantity to the second quantity, to find the indication of cross-section for the blood vessels. Optionally or alternatively, the system includes a blood pressure sensor that measures blood pressure as a function of time for a larger blood vessel than the blood vessels used for the first set of measurements, wherein the signal processor is adapted to find a second quantity indicative of rise time or rate of rise of the blood pressure of the larger blood vessel, and to use a comparison of the first quantity to the second quantity, to find the indication of cross-section for the blood vessels. Optionally, the sensor comprises an optical sensor. Optionally or alternatively, the sensor comprises an ultrasound sensor. Optionally or alternatively, the sensor comprises an electrical impedance sensor. Optionally or alternatively, the sensor has a time resolution better than 20 milliseconds.

There is provided in accordance with an exemplary embodiment of the invention a method for measuring an indication of blood vessel cross section in a subject, the system comprising:
a) making or providing a first set of measurements indicative of a pressure wave in a larger blood vessel and in smaller blood vessels that branch off it, and generating a first signal from the first set of measurements;
b) making or providing a second set of measurements indicative of the pressure wave in the larger blood vessel and in the smaller blood vessels that branch off it, and generating a second signal from the second set of measurements, the larger blood vessel contributing more, relative to the smaller blood vessels, for the first signal, than it does for the second signal;
c) finding time delay between the first signal and the second signal; and
d) using the time delay to find the indication of blood vessel cross section for the smaller blood vessels. Optionally, the larger blood vessel contributes more than the smaller blood vessels to the first signal, and the smaller blood vessels contribute more than the larger blood vessel to the second signal. Optionally or alternatively, the measurements are made on a surface of the subject's body, the first set of measurements respond to the pressure wave with a fall-off down to a first characteristic depth, and the second set of measurements respond to the pressure wave with a fall-off down to a second characteristic depth, less than the first characteristic depth. Optionally, the first characteristic depth is at least 2 times as great as the second characteristic depth. Optionally or alternatively, the first characteristic depth is greater than 5 mm. Optionally or alternatively, the second characteristic depth is less than 5 mm.

In an exemplary embodiment of the invention, the method comprises making the first and second sets of measurements again at another time, finding the time delay at the other time, and using the time delays at both times to find the indication of blood vessel cross section. Optionally, the indication of blood vessel cross section comprises a change in blood vessel cross section or a direction of a change in blood vessel cross section over time. Optionally or alternatively, the method comprises making the first and second sets of measurements again at another place on the subject's body, finding the time delay at the other place, and using the time delays at both places to find the indication of blood vessel cross section. Optionally, the indication of blood vessel cross section comprises a difference in blood vessel cross section, or a direction of difference in blood vessel cross section, between the two places.

In an exemplary embodiment of the invention, the method comprises using the time delay, and separately obtained information about a cross section or expected cross section of the smaller blood vessels, to estimate mean arterial pressure. Optionally or alternatively, the indication of blood vessel cross section comprises one or more of a measure of vasoconstriction, change in vasoconstriction over time, and difference in vasoconstriction in different parts of the body. Optionally, the indication of blood vessel cross section comprises a difference in vasoconstriction between a peripheral and a central part of the body, and the method also includes diagnosing shock, dehydration, or both, from the difference in vasoconstriction.

In an exemplary embodiment of the invention, the indication of blood vessel cross section comprises one or more of a measure of damage to small blood vessels due to a pathological condition, a change in damage to small blood vessels over time, due to a pathological condition, and a difference in damage to small blood vessels in different parts of the body. Optionally, the indication of blood vessel cross section comprises a difference in damage to small blood vessels in different parts of the body, and the method also includes assessing damage due to diabetes, from the difference in damage to small blood vessels between a part of the body damaged by diabetes, and an undamaged part of the body.

In an exemplary embodiment of the invention, using the time delay to find the indication of blood vessel cross-section comprises finding an indication of vasoconstriction of arterioles if the time delay is longer than a critical value, and the critical value is between 40 and 70 milliseconds.

There is provided in accordance with an exemplary embodiment of the invention a method for measuring an indication of blood vessel cross section in a subject, the system comprising:
a) making or providing at least a first set of measurements indicative of a pressure wave in smaller blood vessels that branch off a larger blood vessel, and generating a first signal from the first set of measurements;
b) finding a quantity indicative of rise time or rate of rise of the pressure wave in the smaller blood vessels; and
c) using the quantity indicative of rise time or rate of rise to find the indication of blood vessel cross section for the smaller blood vessels. Optionally, the method comprises making the set of measurements again at another time, finding the quantity indicative of rise time or rate of rise at the other time, and using the quantities at both times to find the indication of blood vessel cross section. Optionally, the indication of blood vessel cross section comprises a change in blood vessel cross section or a direction of a change in blood vessel cross section over time.

In an exemplary embodiment of the invention, the method comprises making the first set of measurements again at another place on the subject's body, finding the quantity indicative of rise rate or rate or rise at the other place, and using the quantities at both places to find the indication of blood vessel cross section. Optionally, the indication of blood vessel cross section comprises a difference in blood vessel cross section, or a direction of difference in blood vessel cross section, between the two places.

In an exemplary embodiment of the invention, the method comprises using the quantity indicative of rise time or rate of rise, and independent information about a cross section or expected cross section of the smaller blood vessels, to estimate mean arterial pressure.

In an exemplary embodiment of the invention, the indication of blood vessel cross section comprises one or more of a measure of vasoconstriction, change in vasoconstriction over time, and difference in vasoconstriction in different parts of the body. Optionally, the indication of blood vessel cross section comprises a difference in vasoconstriction between a peripheral and a central part of the body, and the method also includes diagnosing shock, dehydration, or both, from the difference in vasoconstriction.

In an exemplary embodiment of the invention, the indication of blood vessel cross section comprises one or more of a measure of damage to small blood vessels due to a pathological condition, a change in damage to small blood vessels over time, due to a pathological condition, and a difference in damage to small blood vessels in different parts of the body. Optionally, the indication of blood vessel cross section comprises a difference in damage to small blood vessels in different parts of the body, and the method also includes assessing damage due to diabetes, from the difference in damage to small blood vessels between a part of the body damaged by diabetes, and an undamaged part of the body.

In an exemplary embodiment of the invention, the small blood vessels are less than 1 mm in inside diameter.

In an exemplary embodiment of the invention, the small blood vessels are arterioles.

There is provided in accordance with an exemplary embodiment of the invention apparatus for collecting information about blood vessel cross-section near a surface of the body, comprising:

at least one transmitter and at least one receiver; and

circuitry configured to activate said at least one transmitter and said at least one receiver to collect radiation scattered off two volumes in a body portion, a first volume being configured to include a predominant amount of scattering from arterioles and a second volume being configured to include a predominant amount of scattering from arteries from which said arterioles split off.

There is provided in accordance with an exemplary embodiment of the invention apparatus for processing information about blood vessel cross-section near a surface of the body, comprising:

a signal receiving section which receives two signals, a first signal being including a predominant contribution of scattering from arterioles and a second signal including a predominant contribution of scattering from arteries from which said arterioles split off; and circuitry which processes said signals to detect a difference in time between pulse wave in the two signals.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic drawing of an optical sensor system being used on a surface of a subject's body to measure blood volume or a related parameter as a function of time in an artery, and in smaller arteries and/or arterioles branching off from the artery, according to an exemplary embodiment of the invention;

FIG. 2 is a schematic drawing of an ultrasound sensor system being used on a surface of a subject's body to measure blood volume as a function of time in an artery, and in smaller arteries and/or arterioles branching off from the artery, according to an exemplary embodiment of the invention;

FIG. 3 is a schematic drawing of an electrical impedance sensor system being used on a surface of a subject's body to measure blood volume as a function of time in an artery, and in smaller arteries and/or arterioles branching off from the artery, according to an exemplary embodiment of the invention;

FIG. 4 is a schematic drawing of a laser Doppler system being used to measure blood flow rate as a function of time in an artery near the surface of a subject's body, and in smaller arteries and/or arterioles branching off from the artery, according to an exemplary embodiment of the invention;

FIG. 5 is a flowchart for a method of finding the cross sections of smaller blood vessels branching off from larger blood vessels, or changes in the cross section, for example using the systems shown in FIGS. 1-4, using a time delay in the pulse wave between the larger blood vessels and the smaller blood vessels, according to an exemplary embodiment of the invention;

FIG. 6 is a flowchart for a method of finding the cross sections of smaller blood vessels branching off from larger blood vessels, or changes in the cross sections, for example using the systems shown in FIGS. 1-4, using a rise time or rate of rise in the pulse wave at least for the smaller blood vessels, according to an exemplary embodiment of the invention;

FIG. 7 is a schematic drawing showing a pulse wave as a function of time primarily in a small artery, and primarily in arterioles branching off from the small artery, using a photoplethysmography system similar to the optical sensor system shown in FIG. 1;

FIG. 8 is a flowchart for a method of evaluating damage in small blood vessels that branch off larger blood vessels, in patients with pathological conditions such as diabetes, according to an exemplary embodiment of the invention; and

FIG. 9 is a flowchart for a method of evaluating shock or dehydration in a patient, by finding differences in the cross sections of smaller blood vessels branching off larger blood vessels, for peripheral and central part of the patient's body, and optionally monitoring changes in those differences over time, according to an exemplary embodiment of the invention.

### DESCRIPTION OF SPECIFIC EMBODIMENTS OF THE INVENTION

The present invention, in some embodiments thereof, relates to a method and apparatus for measuring vascular parameters and, more particularly, but not exclusively, to a method and apparatus for monitoring changes in the cross section of small branching arteries and arterioles.

In order to diagnose the diseases mentioned above in the “Field and Background of the Invention” section, as well as hypovolemic shock and dehydration, which are characterized by vasoconstriction of peripheral parts of the body, and in order to monitor their progression, it would be desirable to have a convenient and inexpensive way to continuously monitor the cross sections, and changes in the cross sections, of arterioles and other small blood vessels, but no satisfactory technology for that purpose exists at present.

In principle, one could calculate the diameter of small blood vessels, by combining an optical Doppler measurement of blood flow rate, a photoplethysmography (PPG) sensor to measure blood volume, and an accurate measurement of diastolic pressure inside the blood vessels being examined. But it is difficult to obtain accurate measurements of diastolic pressure in small blood vessels with only external sensors, and besides, optical Doppler measurements of blood flow rate may not be practical for continuous monitoring.

An aspect of some embodiments of the invention relates to finding an indication of blood vessel cross section for small blood vessels that branch off larger blood vessels. The indication can include, for example, one or more of an absolute value, a characteristic value, a change in value and/or a relative value (e.g., as compared to a different location and/or time). Two sets of measurements are made, of a physiological parameter that indicates a pressure wave in the larger and smaller blood vessels, the first set of measurements having a greater contribution from the larger blood vessel, and the second set of measurements having a smaller contribution from the larger blood vessel. For example, the first set of measurements may be dominated by the pressure wave in a larger blood vessel, and the second set of measurements may be dominated by the pressure wave in the smaller blood vessels that branch off it. By measuring a time delay between signals from the two sets of measurements, it is possible to estimate the cross section, or changes in the cross section, of the smaller blood vessels. The time delay may be the time needed for a pressure wave to travel the length of the smaller blood vessels, which depends at least in part on viscous drag in the smaller vessels, and hence on their cross sectional area and shape.

For example, the smaller vessels may be arterioles and the larger ones arteries, possibly arteries form which the arterioles split off.

The measurements of the pressure wave may be, for example, measurements of blood volume in tissue, for example optical measurements, ultrasound measurements, or electrical impedance measurements. The measurements may also be, for example, measurements of blood flow rate, for example laser Doppler measurements. The measurements may be measurements of oxygen or carbon dioxide levels in blood or tissue, for example optical measurements. The two sets of measurements may distinguish larger blood vessels from the smaller blood vessels that branch off them, by penetrating to different characteristic distances beneath the surface of the body. Smaller blood vessels that branch off from larger blood vessels typically extend closer to the surface than the larger blood vessels they branch off from. For example, if optical measurements are used, then larger blood vessels can be measured using a wavelength of light that penetrates further into the tissue, such as near infrared, while smaller blood vessels can be measured using a wavelength of light that does not penetrate as far, for example green light. Both near infrared light, and green light, are suitable for measuring blood volume, because they are both preferentially absorbed by blood over other tissue, and other wavelengths can also be used for this reason. Wavelengths can also be used even if they are not preferentially absorbed by blood, if they provide an indication of the pressure wave in a different way, for example by providing a measure of blood oxygen level or carbon dioxide level. Similarly, if ultrasound measurements are used to measure blood volume, then lower frequencies, which penetrate further into tissue, may be used to measure the larger blood vessels, while higher frequencies are used to measure the smaller blood vessels. In addition, for either optical or ultrasound measurements, the large blood vessels can be measured using a source (light source or ultrasound transducer) that is further away, on the surface of the body, from the detector, while the smaller blood vessels, closer to the surface of the body, can be measured using a source that is closer, along the surface of the body, to the detector, so that the signal is dominated by light or ultrasound that has not penetrated very far beneath the surface. Similarly, for electrical impedance measurements, electrodes can be placed further from each other on the surface of the body, to measure larger blood vessels, which are deeper in the body, and closer to each on the surface of the body, to measure smaller blood vessels, which are closer to the surface of the body.

In some embodiments of the invention, the first set of measurements is made using a sensor placed next to a blood vessel close to a surface, large enough to be visible to the naked eye, or through an endosope, and the second set of measurements is made using a sensor placed in a nearby area of the surface where there is no large blood vessel, visible to the naked eye or through an endoscope, near the surface, so the measurements will be dominated by smaller blood vessels that branch off the larger blood vessel. This method may be particularly useful for measurements made of the surfaces of internal organs, for example endoscopically or during surgery, for which relatively large blood vessels are likely to be visible on the surface.

In an exemplary embodiment of the invention, domination means at least 50%, at least 60%, at least 70% or intermediate or smaller values, as compared to the contribution of the other pulse wave. It is noted that it is possible to collect measurements form overlapping volumes and subtract the signals, so as to provide a signal which is dominated by one or the other effect and/or volume. In addition, signal processing methods, such as frequency based filtering may be used to process a signal so that it is dominated by a desired indication of pressure wave (e.g., pulse wave).

In an exemplary embodiment of the invention, the signals are analyzed using, for example, a set of thresholds, a table of thresholds and/or ranges, possibly overlapping, which match a value with a vasoconstriction condition or other desired parameter. Optionally or alternatively, other processing methods are used, for example, a neural network, an expert system and/or an algorithm. In some cases, the signal or signal pair or relationship between them (e.g., time delay or change in time delay) are translated directly into a value for display.

In an exemplary embodiment of the invention, detection of signals is gated and/or coherent detection is used. For example, signals are sensed and/or processed if they fall within a time window relative to a local sensing of a pressure wave (e.g., in an artery or using a different sensor) and/or an estimation of arrival thereof. Optionally or alternatively, signals which do not match an expected behavior of a pressure wave (e.g., not at a physiological pulse rate), are ignored.

In an exemplary embodiment of the invention, a user input is provided to the processing circuitry, for example, to input temperature, medication and/or other information which can be used in processing the signals and/or analyzing the results of such processing. Optionally or alternatively, such input is from sensors (e.g., temperature) and/or is provided by computer communication.

An aspect of some embodiments of the invention relates to finding an indication of blood vessel cross sections, where possibly measurements of only one size of blood vessels is needed. In this method, optionally only one set of measurements is made, of a pressure wave in smaller blood vessels, and a quantity indicating a rise time or rate of rise of the pressure wave is found. The rise time of the pressure wave has been found to be increased by vasoconstriction, so can be used to provide an indication of the blood vessel cross section. Again, the measurements can comprise using optical, ultrasound or electrical impedance measurements to measure blood volume, or laser Doppler measurements to measure blood flow rate. Optionally, the measurements are also made on larger blood vessels, to provide a reference case, where the viscous drag is relatively small, for comparison.

Either of these methods can be used to assess various medical conditions. Vasoconstriction, which is a reversible decrease in blood vessel diameter, specifically for arterioles, can be an indication of shock, or dehydration. Pathological conditions such as diabetes, or atherosclerosis, can cause long term irreversible narrowing of small blood vessels, or changing of cross section shape, and can be diagnosed or monitored using these methods. For these pathological conditions, narrowing of the blood vessels may be associated with a decrease in time delay between larger and smaller blood vessels, or a decrease in rise time in smaller blood vessels, if the blood vessel walls also become stiffer due to the pathological condition, but measuring these quantities can still be used to distinguish damaged small blood vessels, from healthy ones.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

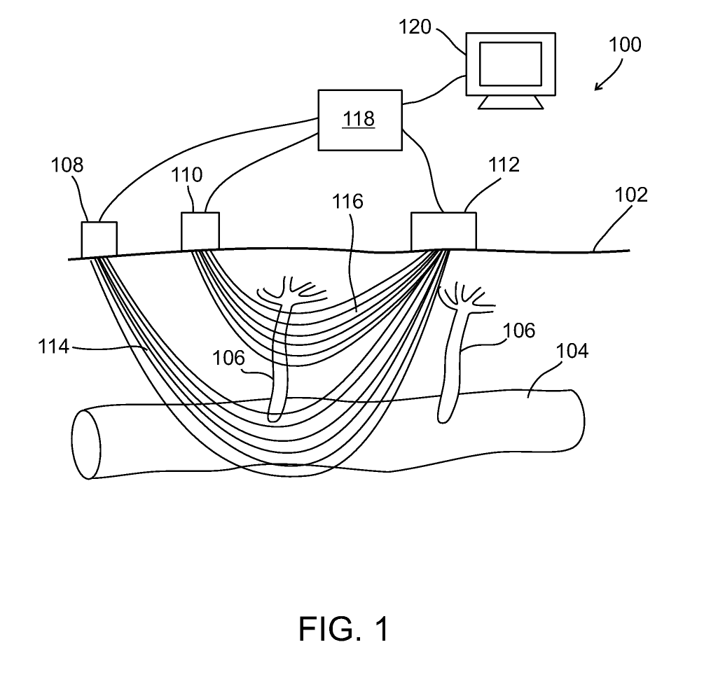

Referring now to the drawings, FIG. 1 illustrates an optical sensor system 100, for example a photoplethysmography (PPG) system, used to measure blood volume, or a related parameter such as blood or tissue oxygen level or carbon dioxide level, as a function of time in a region of a surface 102 of a subject's body, according to an exemplary embodiment of the invention. Optionally, surface 102 is the subject's skin, and sensor system 100 is non-invasive. Alternatively, system 100 can be used on an internal surface of the subject's body, for example on a surface of an internal organ during surgery, or in an endoscopic procedure, for example in the nasal passage, in the gastrointestinal tract, in the ear, or in the urethra. A blood vessel 104, at some distance beneath surface 102, has smaller blood vessels 106 branching off it. When a blood vessel has smaller blood vessels branching off it, the smaller blood vessels often come closer to the surface than the larger blood vessel, especially when the surface is the skin. For example, blood vessel 104 is a relatively small artery, about 7 mm beneath the surface, and vessels 106 are arterioles, which come closer to the surface than vessel 104, for example within 2 mm, 3 mm or 4 mm of the surface.

Light sources 108 and 110, aimed into surface 102 and optionally in contact with surface 102, illuminate the blood vessels, and light scattered from the blood vessels is detected by detector 112. Light source 108 produces light of a relatively long wavelength, for example near infrared light, that can penetrate deeply enough into body tissue to reach the depth of blood vessel 104, while light source 110 produces light of a shorter wavelength, for example green light, which largely does not penetrate the tissue as far as blood vessel 104, but mostly illuminates smaller blood vessels 106, that are closer to the surface. In this way, detector 112 can generate a first signal to which blood vessel 104 makes a substantial contribution, and a second signal to which blood vessel 104 makes a smaller contribution, if any, and smaller blood vessels 106 make a relatively larger contribution.

For example, the light from light source 108 penetrates to a characteristic fall-off distance of 3 mm, or 5 mm, or 10 mm, or more than 10 mm, or less than 3 mm, or an intermediate distance. A characteristic fall-off distance in tissue for light from light source 110 is smaller than the characteristic fall-off distance for light from light source 108, for example by a factor of at least 1.3, or at least 1.5, or at least 2, or at least 3, or at least 5. For example, the light from light source 110 penetrates to a characteristic fall-off distance of 1 mm, or 2 mm, or 3 mm, or 5 mm into the tissue, or a greater, smaller, or intermediate distance. Optionally, one or both of light sources 108 and 110 is an LED, or a laser diode. In some embodiments of the invention, light sources 108 and 110 comprise a single light source, which produces two different wavelength bands of light, a longer wavelength band of light which penetrates more deeply into the tissue, and a shorter wavelength band of light which penetrates less deeply. In some embodiments of the invention, the light source or separate light sources produce three or more wavelength bands of light, which penetrate into the tissue respectively a shorter distance, one or more different intermediate distances, and a longer distance. Using three or more wavelength bands may provide more accurate results for time delay as a function of penetration distance, because there is some redundancy. Additional wavelength bands may also be used to measure different parameters, for example both blood volume, and blood oxygenation level, which may provide more accurate results.

In addition to, or instead of, using a wavelength range for light source 108 that penetrates tissue more deeply than a wavelength range used for light source 110, the light detected from light source 108 will come from a deeper layer of tissue than the light detected from light source 110, if light source 108 is located further away from its detector than light source 110 is. Light detector 112 is optionally positioned on surface 102, close enough to light source 108 that it can detect a substantial amount of light from light source 108 that scatters from tissue at the depth of blood vessel 104, but not so close to light source 108 that light from light source 108 scattering from a shallower depth in the tissue, for example at the depth of blood vessels 106, overwhelms the light scattered from tissue at the depth of blood vessel 104. For example, light detector 112 is located at a distance from light source 108 equal to 0.5 times a characteristic fall-off distance in tissue of the light from light source 108, or equal to the characteristic fall-off distance, or equal to 2 times the characteristic fall off distance, or 3 times the characteristic fall off distance, or equal to 3 mm, or 5 mm, or 10 mm, or 20 mm, or 30 mm, or equal to a smaller, greater, or immediate distance. Optionally, light detector 112 is also used to detect light from light source 110 that scatters from tissue at a shallower depth, or a separate light detector is used for that purpose. Light detector 112, or a separate light detector if one is used, is located close enough to light source 110 so that it detects a substantial amount of light from light source 110 that scatters from tissue at the depth of blood vessels 106, but not so close that light scattered from a shallower depths overwhelms the light scattered from tissue at the depth of blood vessels 106. For example, light detector 112, or a separate light detector used for light source 110, is located at a distance from light source 110 equal to 0.5 times a characteristic fall-off distance in tissue of the light from light source 110, or equal to the characteristic fall-off distance, or equal to 2 times the characteristic fall off distance, or 3 times the characteristic fall off distance, or equal to 0.5 mm, or 1 mm, or 2 mm, or 5 mm, or 10 mm, or equal to 1 times, 1.5 times, 2 times, 3 times, 5 times or 10 times the distance between light source 108 and light detector 112, or equal to a smaller, greater, or immediate distance. If there are three or more light sources producing light of different wavelengths which penetrate different distances into the tissue, then the light sources producing the more deeply penetrating light are optionally located further from the detector, or their individual detector, than the light sources producing the less deeply penetrating light.

When system 100 operates, light source 108 produces light 114, directed into the tissue beneath surface 102, which scatters relatively more from blood vessel 104, and relatively less from smaller blood vessels 106, and is detected by detector 112, while light source 110 produces light 116, directed into the tissue beneath surface 102, which scatters relatively more from smaller blood vessels 106, and relatively less from blood vessel 104, and is detected by light detector 112, or by a different light detector as noted above. It should be understood that “relatively more” and “relatively less,” mean that the ratio of light scattered from blood vessel 104 to light scattered from blood vessels 106 is greater for light produced by light source 108 and detected by light detector 112, than it is for light produced by light source 110 and detected by light detector 112. Optionally, the ratio is 1.2 times as great, or 1.5 times as great, or 2 times as great, or 5 times and great, or 10 times as great, or a smaller, greater, or intermediate number of times as great. Optionally, more of the light produced by light source 108 and detected by light detector 112 is scattered by blood vessel 104 than by blood vessels 106, for example 1.2 times as much, or 1.5 times as much, or 2 times as much, or 5 times as much, or 10 times as much, or a smaller, greater, or intermediate number of times as much. Optionally, more of the light produced by light source 110 and detected by light detector 112 is scattered by blood vessels 106 than by blood vessel 104, for example 1.2 times as much, or 1.5 times as much, or 2 times as much, or 5 times as much, or 10 times as much, or a smaller, greater, or intermediate number of times as much.

Optionally, light sources 108 and 110 illuminate the tissue beneath surface 102 simultaneously, and light detector 112 distinguishes between light from light source 108 and light from light source 110 by using filters, or using two detectors that are each sensitive to wavelengths from a different one of the light sources. Alternatively, light coming from light source 108 is distinguished from light coming from light source 110 by multiplexing, i.e. the light sources are alternately turned on and off, with only one of the light sources on at a given time. However, if such multiplexing is used, it may be advantageous to do it rapidly enough, for example with on and off times of several milliseconds or less, so that a time delay between signals from the two light sources, that is only a few tens of milliseconds, can be accurately measured, as will be explained below.

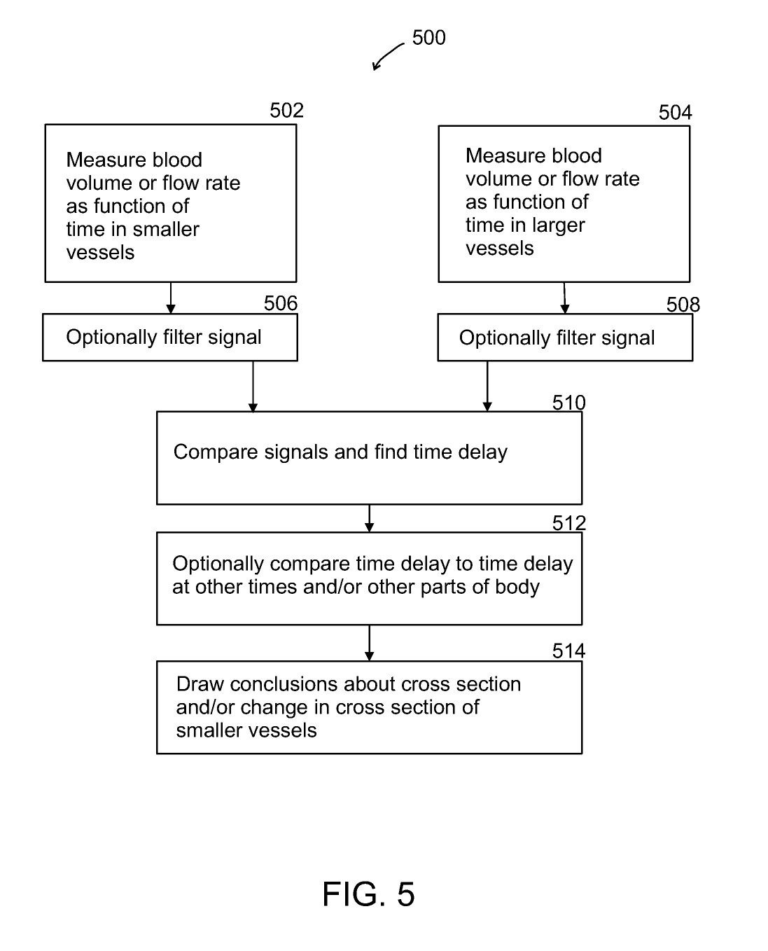

The light from light source 108 detected by light detector 112, scattered relatively more from blood vessel 104 and less from smaller blood vessels 106 than the light from light source 110 is, provides a measure of the volume of blood or a related parameter in blood vessel 104, in the vicinity of the light sources and detector, as a function of time. The light from light source 110 detected by light source 112, scattered relatively more from blood vessels 106, and less from blood vessel 104, provides a measure of the volume of blood or a related parameter in blood vessels 106, in the vicinity of the light sources and detector, as a function of time. Two signals produced by detector 112, one of light produced by light source 108 and one of light produced by light source 110, are sent to a controller 118, for example a computer or dedicated circuitry. Controller 118 compares the two signals, and, as will be described below in the description of FIG. 5, uses the signals to obtain information about the cross section of blood vessels 106, or about a change in the cross section of blood vessels 106, or a difference in the cross section in different parts of the body. As used herein, “information about the cross section” refers not only to the cross sectional area of the blood vessel lumen, but also the shape of the cross section, and other parameters that affect the time delay, such as a stiffness of the blood vessel walls. Alternatively or additionally, other parameters of the blood vessels may be found, for example the mean arterial pressure may be found if there is other information about the cross sectional area and shape.

Even if the signals from light produced by light source 108 and light produced by light source 110 are not dominated respectively by scattering from blood vessel 104 and scattering from blood vessels 106, in some embodiments of the invention the fact that scattering from blood vessel 104 and scattering from blood vessels 106 make different relative contributions to the signals, allows controller 118 to separate the contribution from blood vessel 104 from the contribution from blood vessels 106, and to create two output signals that, subject to noise and other limitations of the data, represent only or primarily scattering from blood vessel 104 and blood vessels 106 respectively. Optionally, controller 118 uses those two output signals, instead of or in addition to the two signals of light produced by light source 108 and light produced by light source 110, to find the information about the cross section or change or difference in cross section of blood vessels 106.

Optionally, controller 118 is connected to an I/O device 120, such as a display screen, a printer, a touch screen, a keyboard, and/or a mouse, that allows users to see the results of calculations done by controller 118. Optionally, controller 118 also controls and/or detects when light sources 108 and 110 are turned on. Optionally, a user can use the input features of I/O device 120 to turn system 100 on, and/or to control parameters used by controller 118 in analyzing the signals from light detector 112, optionally using a graphic user interface. Controller 118, I/O device 120, and the light sources and detector need not be physically located in the same room, but may be remote from each other, connected by communications links. For example, I/O device may be a cell phone or a Bluetooth device, used to monitor a patient remotely. I/O device 120 may also be located next to the patient, or even on a device worn by the patient, such as a bracelet with a display screen, so medical personnel can easily read off data from it when examining the patient.

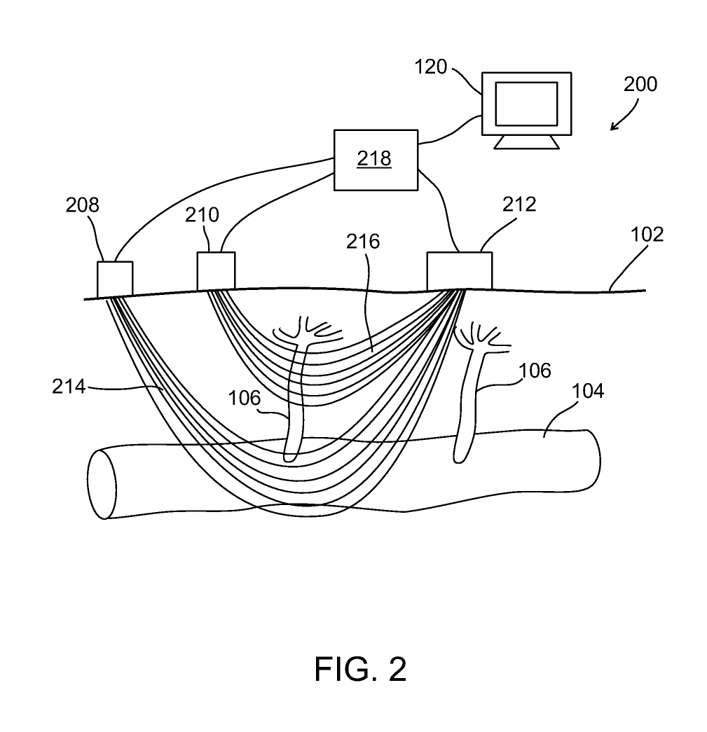

It should be understood that elements with a function attributed to controller 118, for example A/D converters, or CPUs, may also be located in detector 112, and this is true also for the systems shown in FIGS. 2-4, and the detectors or receivers, and controllers, in those systems. Alternatively, such elements may be considered part of controller 118, even if they are housed in a same physical unit as detector 112. Digital sensors may be used as well (e.g, sensors with a digital output, optionally time encoded). Optionally a clock is provided. In general, controller 118, and the other controllers in FIGS. 2-4, need not be a single physical unit, but are optionally distributed in a plurality of different places or combined with different pieces of hardware. In some specific examples, sensing may be by a disposable or low cost element, which transmits signals (wired or wirelessly), optionally partially processed to a processor, such as a mobile telephone (which optionally controls the sensing), or to (directly or indirectly) a server, for example, a hospital server or a remote server reached over the internet, a cellphone network or other network.

In another example, a wrist worn device or pendant is used by a user to collect process and optionally display data. Optionally or alternatively, the data is displayed via a remote or local server.

In another example, processing and display is performed on a co-located mobile device, such as a smart telephone, with display and/or acquisition optionally provided by an app (e.g., a software on the smart phone).

Data may be processed and/or displayed in parallel at multiple locations and/or at multiple qualities and/or with multiple uses (e.g., one location shows realtime values, another shows trends and a third can show an interpretation). Example locations are with the patient, at a doctor's computer and on the device itself (e.g., on a display thereof).

Such remote processors can also be provided with information to assist in analyzing the collected data, such as patient history.

Display can be, for example, visual displays and/or audio displays. Optionally or alternatively, alters are provided, for example, if certain thresholds are crossed.

Various such thresholds and other data is optionally stored in memory, for example, of the device and/or of a server, for example, a server than monitors multiple patients.

It should also be understood that more than one system such as system 100, or elements of more than one such system, may be used on a same subject. For example, different types of sensors, such as those in FIGS. 1-4, may be used together, with different controllers, or with a single controller that performs the control functions for all of the sensors.

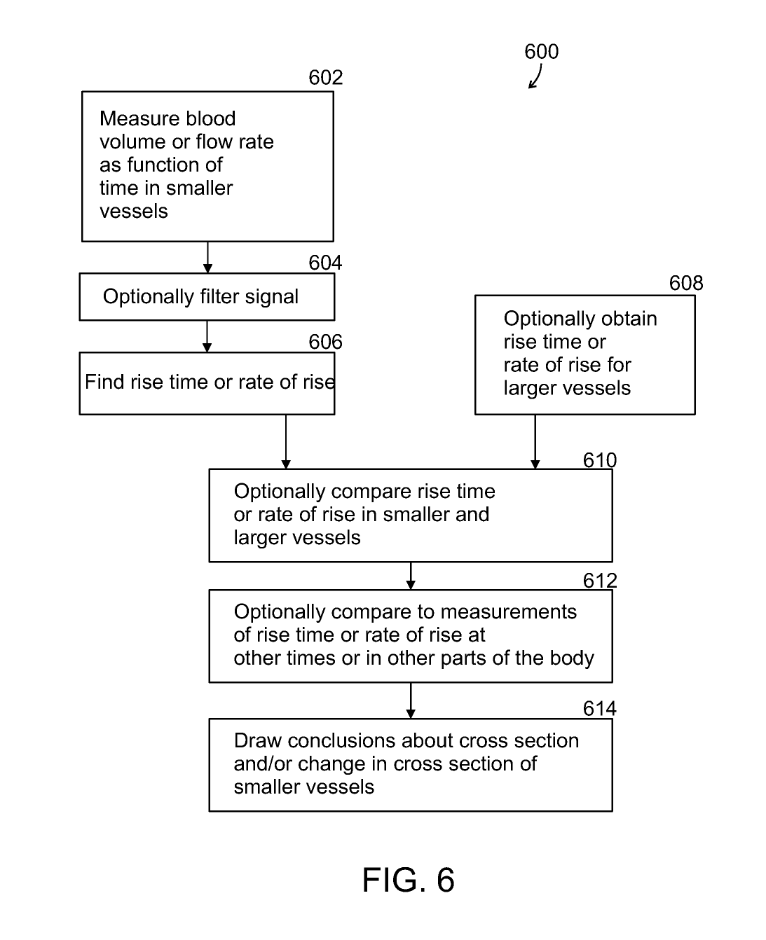

In some embodiments of the invention, including for example some of the embodiments described below in the description of FIG. 6, only a single light source 110 is used, and light detector 112 produces only a single signal, of light primarily scattered from small blood vessels 106. In these embodiments, controller 118 finds information about the cross section, difference in cross section or change in cross section of blood vessels 106, using only that one signal as a function of time from light detector 112, optionally in combination with signals from other sensors.

Light scattered from tissue provides a measure of the blood volume in the scattering region, if the light is of a wavelength or band of wavelengths that is absorbed and/or scattered at a rate different from the rest of the tissue, and this is true of the light produced by light sources 108 and 110. For example, the light produced by light source 110 is optionally in an absorption band of oxyhemoglobin, if system 100 is designed to be used for arteries, or deoxyhemoglobin if system 100 is designed to be used for veins. The light produced by light source 108 is optionally in a wavelength range, in the near infrared, that is absorbed by water with an absorption length on the order of 1 cm or a few cm, for example between 0.9 and 1.4 μm, so would be preferentially absorbed by blood, which has a higher percentage of water than the surrounding tissue, but would not be almost completely absorbed before it reaches blood vessel 104.

In some embodiments of the invention, light sources 108 and 110 use wavelengths that are not preferentially absorbed by blood over other tissue, but that are preferentially absorbed by oxyhemoglobin over deoxyhemoglobin, or vice versa, or that are absorbed by carbon dioxide, for example in the infrared at 2.15 μm or 4.2 μm. Such wavelengths are used by optical pulse oximeters, and by optical capnometers. In this case, the signal produced need not be a measure of blood volume, but may be a measure of oxygen level or carbon dioxide level in the blood, and in tissue. Since oxygen levels and carbon dioxide levels in the blood, and in tissue, may vary periodically over the cardiac cycle and over the breathing cycle, they may be used, as an alternative to blood volume, to find a time delay between the signals, as described in FIG. 5, or to find a quantity related to rise time or rate of rise in one or both of the signals, as described in FIG. 6.

In some embodiments of the invention, there are a plurality of different light sources 108 located at different distances from detector 112, and/or a plurality of different light sources 110 located at different distances from detector 112, and/or a plurality of different detectors 112 located at different distances from light sources 108 and 110. The distance between light source 108 and detector 112, and/or the distance between light source 110 and detector 112, can then be optimized, by looking at the signal for each distance between the light sources and the detector, and optionally only using the signals that work best. Optionally, there are also a plurality of light source and detectors pairs at the same separation distance, but located at different places, and the location can be optimized by looking at the signal from each pair, and optionally only using the signal that works best. Some locations may work better than other locations because, for example, the light source and detector are positioned better with respect to blood vessels that are suitable for measuring vasoconstriction, or that are suitable for detecting narrowing of blood vessels due to a pathological condition, such as diabetes, that may only affect blood vessels in some locations. For example, a linear array of, for example, between 2 and 10 LEDs may be used, with optimization being used to find a pair of LEDs which provide a maximum difference in time delay, minimal noise, stability and/or other signal consideration. The selection of LEDS may reflect, for example, the relative depths of artery-dominated and arteriole-dominated tissue volumes. Optionally or alternatively, sensors or sources arranged perpendicular to a line connecting the sensor and source and/or a two dimensional array of sensors and/or of sources may be used to assist in selecting different regions with a useful signal.

In an exemplary embodiment of the invention, the sensed volumes are, for example, each less than 10 cubic mm, 3 cubic mm, 1 cubic mm or intermediate or greater volumes, the shape of the volume sis optionally selected to match physiological considerations, for example, being thin, for example, having a thickness (e.g., from which most or significant part of the signal is scattered) of less than 5 mm, less than 3 mm, less than 1 mm.

FIG. 2 shows a system 200, similar to system 100, but using ultrasound scattering from tissue, instead of light scattering from tissue, to measure blood volume. An ultrasound transducer 208 produces ultrasound of a lower frequency that penetrates further into the tissue beneath surface 102 than ultrasound of a higher frequency produced by an ultrasound transducer 210. For example, the lower frequency ultrasound may be between 0.3 and 1.5 MHz, and the higher frequency ultrasound may be between 1.5 and 6 MHz. An ultrasound receiver 212 detects and distinguishes between ultrasound of the higher and lower frequencies that scatters respectively from smaller blood vessels 106 and larger blood vessel 104. Alternatively, separate receivers are used for the two frequencies or frequency ranges. Similar to system 100, the distance between receiver 212, and transducers 208 and 210, are optionally chosen so that relatively more of the ultrasound from transducer 208, that is received by receiver 212, is scattered from blood vessel 104, while relatively more of the ultrasound from transducer 210, that is received by receiver 212, is scattered from blood vessels 206. The penetration distances, and distances between the light sources and light detector, described for system 100, optionally apply also for the penetration distances, and the distances between the ultrasound transducers and receivers, for system 200.

Receiver 212 may distinguish between ultrasound of the lower frequency produced by transducer 208, and ultrasound of the higher frequency produced by transducer 210, by electronic filtering, or by multiplexing, for example.

Optionally, lower frequency ultrasound 214, emitted from transducer 208 and detected by receiver 212, provides a measure of the blood volume of blood vessel 104, by producing an ultrasound image of blood vessel 104. The image is produced by a controller 218, from the signals produced by receiver 212, for the lower frequency ultrasound received. Additionally or alternatively, the lower frequency ultrasound may provide a measure of the blood volume in larger blood vessels, such as blood vessel 104, if it has a wavelength that corresponds to the length of the larger blood vessel. For example, if it is known that blood vessel 104 has a length L, for example 3 mm, then the lower frequency ultrasound can be chosen to have a wavelength close to 2 L, for example 0.27 MHz, which will preferentially scatter from blood vessels of that length, and the scattering strength may be proportional, for example, to the volume of the blood vessel. If a given frequency is being used for transducer 208, with a wavelength equal to twice a typical length of larger blood vessels such as blood vessel 104, then system 200 can be moved over surface 102 until a relatively high scattering amplitude is observed, indicating a nearby blood vessel that is resonant with that frequency. Keeping system 200 at that location, the scattering amplitude as a function of time, for ultrasound produced by transducer 208, will provide a measure of the volume of that blood vessel as a function of time.

Optionally, higher frequency ultrasound 216, emitted from transducer 210 and detected by receiver 212, provides a measure of the blood volume of blood vessels 106, because it is of a frequency that is resonantly scattered by blood vessels 106, if most of them are about half a wavelength long. For example, arterioles are typically close to 1 mm long, so ultrasound of about 0.8 MHz, for which a wavelength is about 2 mm, may preferentially scatter from the arterioles. The amount of scattering may be proportional, for example, to the volume of the arterioles, providing a measure of the blood volume in arterioles.

Alternatively, if a frequency much higher than 0.8 MHz is used for the higher frequency ultrasound, it may provide a measure of the blood volume of blood vessels 106, due to Rayleigh scattering from erythrocytes in the blood vessels. As described by Emilie Franceschini, Bruno Lombard, and Joel Piraux, “Ultrasound characterization of red blood cells distribution: a wave scattering simulation study,” Journal of Physics: Conference Series 269 (2011) 012014, erythrocytes scatter ultrasound at frequencies greater than about 10 MHz, so the scattering for ultrasound from tissue, for frequencies above 10 MHz, may provide a measure of the volume of blood in the scattering region. Scattering may be limited to or dominated by scattering from smaller blood vessels 106, if the ultrasound is of a high enough frequency that it cannot penetrate as far as larger blood vessel 104 and back to receiver 212, or if relatively little of the ultrasound power can penetrate that far, for example at frequencies of about 30 MHz. Even at lower frequencies, for example below 20 MHz, where ultrasound may easily penetrate as far as large blood vessel 104, relatively little of the scattered ultrasound received by receiver 212 may come from that deep in the tissue, if the separation distance between ultrasound transducer 210 and receiver 212 is less than the depth of blood vessel 104. Although ultrasound with even higher frequency, 50 MHz or 60 MHz, scatters even more efficiently from erythrocytes, such high frequency ultrasound may not be able to penetrate even far enough to reach blood vessels 106, so frequencies of 10 MHz to 40 MHz may work best, for using scattering from erythrocytes as a way of measuring blood volume in blood vessels 106, although frequencies of 4 to 10 MHz, or 40 to 60 MHz, can also be used.

Even if the signals from ultrasound produced by ultrasound transducer 208 and ultrasound produced by ultrasound transducer 210 are not dominated respectively by scattering from blood vessel 104 and scattering from blood vessels 106, in some embodiments of the invention, the fact that scattering from blood vessel 104 and scattering from blood vessels 106 make different relative contributions to the signals, allows controller 118 to separate the contribution from blood vessel 104 from the contribution from blood vessels 106, and to create two output signals that, subject to noise and other limitations of the data, represent only or primarily scattering from blood vessel 104 and blood vessels 106 respectively. Optionally, controller 118 uses those two output signals, and/or the two original signals of ultrasound produced by ultrasound transducer 208 and ultrasound produced by ultrasound transducer 210, to find information about the cross section or change or difference in cross section of blood vessels 106.

Similarly to system 100, controller 218 is optionally connected to an I/O device 120, which provides a user interface similar to that described for system 100. Also, similarly to system 100, in some embodiments of the invention the signal depending on the blood volume of blood vessel 104 is not used, but only a signal depending on the blood volume of blood vessels 106, optionally in combination with other data, to find the information about the cross section or change or difference in cross section of blood vessels 106, or other parameters of blood vessels 106.

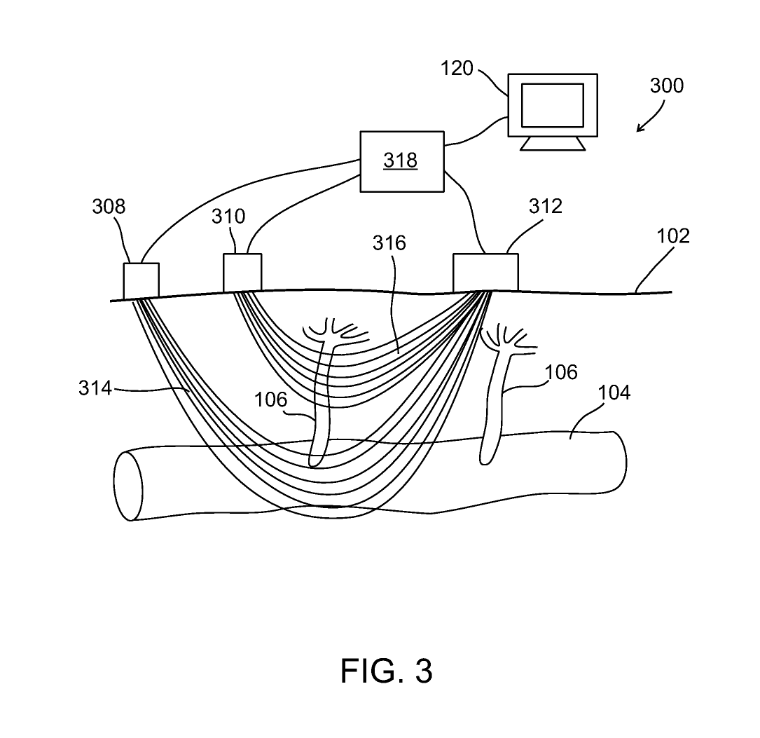

FIG. 3 shows a system 300, similar to system 100 and system 200, but using electric impedance measurements of tissue, to measure blood volume. Since blood generally has a higher conductivity than other body tissue, electrical impedance measurements can be used to measure the blood content of tissue, and in particular to measure changes in blood volume over time, for example due to pressure waves, even if calibration is lacking for measuring the blood volume absolutely. System 300 comprises electrode units 308, 310, and 312, connected to a controller 318 to also includes an electric power supply. Optionally, a separate power supply is used, optionally controlled by controller 318. When system 300 operates, a voltage is applied between two electrode units that are relatively far apart, for example electrode units 308 and 312 in FIG. 3, producing an electric field 314 that has a relatively large fall-off distance into the tissue beneath surface 102, comparable to or greater than the depth of blood vessel 104. The applied voltage produces a current along electric field 314 between electrode units 308 and 312, a substantial part of which passes through blood vessel 104, so the impedance, the ratio of voltage to current, between electrode units 308 and 312, varies significantly with variations in the blood volume of blood vessel 104, and can be used to measure pressure waves in blood vessel 104.

It should be understood that, as is common in bio-impedance measurements, each electrode unit optionally comprises a voltage measuring electrode and a current carrying electrode, adjacent to each other but electrically isolated from each other, and the impedance is optionally measured by measuring the voltage between the voltage measuring electrodes of electrode units 308 and 312, when a known current passes between the current carrying electrodes of electrode units 308 and 312. Using separate voltage measuring and current carrying electrodes has the potential advantage that the voltage measurement will then largely exclude possibly large voltage drops associated with the current passing through the high impedance of the epidermis, which is not of interest for measuring blood volume. Although it is also possible to measure impedance by using a known voltage and measuring the current, for safety reasons it is more usual, with live subjects, to hold the current fixed and measure the voltage. Also for safety reasons, the impedance measurement is preferably done at frequencies of at least a few kHz or a few tens of kHz, for example at about 20 kHz. However, the frequency used is optionally well below 100 kHz, since at frequencies of about 100 kHz and higher, substantial current goes through the cell membranes, which act like capacitors, and the resulting impedance measurement may be less sensitive to blood volume.

A voltage is also applied between electrode unit 310 and electrode unit 312, or between electrode unit 310 and a separate electrode unit not shown in FIG. 3, which are closer together on surface 102 than electrode unit 308 and electrode unit 312. This produces an electric field 316, and a current running along electric field 316, which does not penetrate as far into the tissue as electric field 314, and in particular may fall off significantly at the depth of blood vessel 104, more than electric field 314 does. As a result, the impedance measured between electrode units 310 and 312 is relatively less sensitive to the blood volume of blood vessel 104, than the impedance measured between electrode units 308 and 312, and relatively more sensitive to the blood volume of smaller blood vessels 106, which are closer to surface 102.

Controller 318 uses the voltages and currents to calculate two impedance signals as a function of time, a signal representing the impedance between electrode units 308 and 312, and a signal representing the impedance between electrode units 310 and 312, or between electrode unit 310 and a separate impedance unit. If electrode unit 312 is used for both impedance signals, and if the measurements are made simultaneously, then optionally the two impedance signals are distinguished by using different frequencies for them. Alternatively, multiplexing may be used to distinguish the two signals. Optionally, the signals have a time resolution short enough to measure a time delay of a few tens of milliseconds with reasonable accuracy, for example the time resolution is about 1 millisecond. In this case, the frequencies used for the two impedance signals optionally differ by at least 1 kHz, so the two signals can be distinguished from each other over a time of 1 millisecond, but optionally do not differ by so much that they measure significantly different biological phenomena, for example extracellular impedance and intracellular impedance. For example, using 20 kHz for one signal and 21 kHz for the other signal satisfies these criteria.

Even if the signals from the impedances measured between electrode units 308 and 312, and between electrode units 310 and 312, are not dominated respectively by scattering from blood vessel 104 and scattering from blood vessels 106, the fact that, in some embodiments of the invention, scattering from blood vessel 104 and scattering from blood vessels 106 make different relative contributions to the signals, allows controller 118 to separate the contribution from blood vessel 104 from the contribution from blood vessels 106, and to create two output signals that, subject to noise and other limitations of the data, represent only or primarily scattering from blood vessel 104 and blood vessels 106 respectively. Optionally, controller 118 uses those two output signals, and/or the two original signals of impedance measured between electrode units 308 and 312 and between electrode units 310 and 312, to find information about the diameter or change or difference in diameter of blood vessels 106.

Similarly to systems 100 and 200, controller 318 is optionally connected to an I/O device 120, which provides a user interface similar to that described for system 100. Also, similarly to systems 100 and 200, in some embodiments of the invention the signal depending on the blood volume of blood vessel 104 is not used, but only a signal depending on the blood volume of blood vessels 106, optionally in combination with other data, to find the information about the cross section or change or difference in cross section of blood vessels 106.

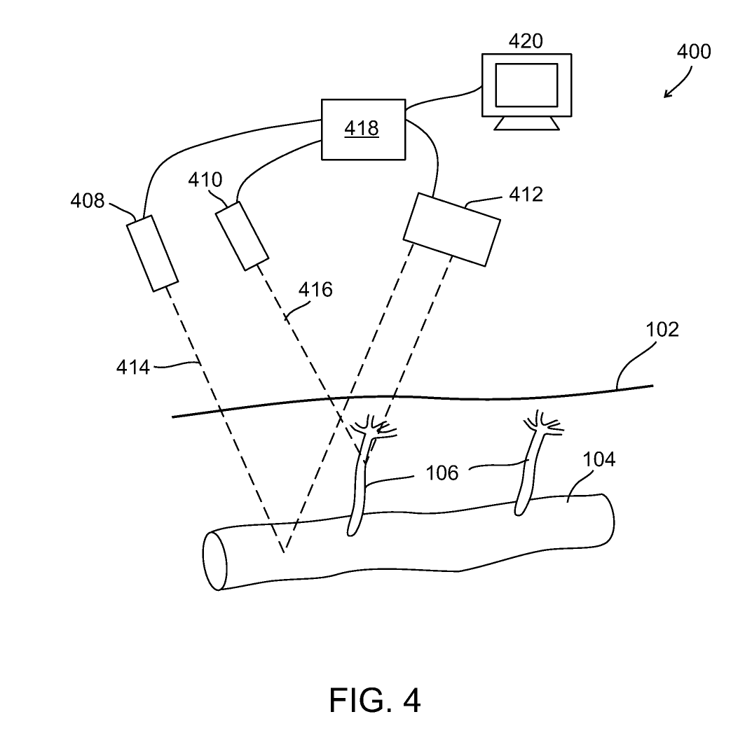

FIG. 4 shows a system 400 that uses laser Doppler measurements to measure a blood flow rate in blood vessel 104, and in one or more of blood vessels 106. A laser Doppler system 408 scatters one or more laser beams from moving erythrocytes in blood vessel 104, which are received by a detector 412. A laser Doppler system 410 scatters one or more laser beams from moving erythrocytes in one or more of blood vessels 106, which are received by detector 412, or by a separate detector. Optionally, laser Doppler system 408 uses a wavelength of light that penetrates further into the tissue beneath surface 102, so it can reach blood vessel 104 while remaining coherent, than the light of laser Doppler system 410, which only has to penetrate as far as blood vessels 106. Alternatively, they use the same wavelength. Detector 412 optionally uses the different wavelengths to distinguish the signals from the two laser Doppler systems. Alternatively, detector 412 uses multiplexing to distinguish the signals.

Detector 412 sends signals from the two laser Doppler systems to a controller 418, which uses the signals to calculate a flow rate of blood in blood vessel 104, and a flow rate of blood in one or more of blood vessels 106, as a function of time. Optionally, because there may be many blood vessels 106 oriented in many different directions, and it may be difficult to determine the direction of orientation of a particular blood vessel 106 that is being measured, laser Doppler system 410 makes a 2D or 3D measurement of blood vessel 106, so that the flow rate can be found as a function of time, by controller 418, even if the orientation of the vessel is not known. Optionally this is also done by laser Doppler system 408 for blood vessel 104.

Because blood flow rate, like blood volume, varies in blood vessels 104 and 106 over time, depending on the pressure, the signals of blood flow rate, like the signals of blood volume, can be used to measure the pressure wave in blood vessels 104 and 106, and hence can be used by controller 418 to find information about the cross section, change in cross section or difference in cross section of blood vessels 106, as will be described below in the descriptions of FIGS. 5 and 6. In some embodiments of the invention, as noted above, only the signal from blood vessels 106 is needed, and for those embodiments, the flow speed or velocity in blood vessel 104 need not be measured.

FIG. 5 shows a flowchart 500, for a method of using measurements for one or more larger blood vessels and for smaller blood vessels that branch off the larger vessels, to find information about the cross section of the smaller vessels, and/or about changes in the cross section over time, and/or about differences in the cross section between different parts of the body. The measurements can be any measurement in the larger and smaller blood vessels, as a function of time, that depends on the pressure, and provides an indication of a pressure wave in those blood vessels, for example blood volume, flow rate, or oxygen level or carbon dioxide level in blood or tissue. The term “pressure wave” as used herein includes the variation in blood pressure in arteries due to the cardiac cycle, as well as a variation in blood pressure in veins due to motion of the subject's body, or any other cause of short-term temporal variation of pressure in blood vessels.

At 502, a measurement is made of blood volume or blood flow rate as a function of time in the smaller blood vessels, and at 504, simultaneously with 502, or with a known delay, a measurement is made of blood volume or blood flow rate as a function of time in the larger blood vessel, using any of the methods described in FIGS. 1-4, for example. At 506, the signal from the smaller vessels is optionally filtered to remove noise, optionally by low-pass filtering, and at 508 the signal from the larger blood vessel is optionally filtered to remove noise, optionally by low-pass filtering. The low-pass filtering removes high frequency noise from the signals, but optionally the filtering is not so strong that the overall shape of the signal on the time scale of the pressure wave is greatly distorted. In particular, the filtering is not so strong as to introduce substantial errors in a time delay between the two signals. For example, frequencies up to 5 times the heart beat frequency, or up to 10 times the heart beat frequencies, are not filtered very much, but higher frequencies are. Optionally, very low frequency components, for example at frequencies below the frequency of the heart beat, are also filtered out, to detrend the data, or the data is detrended in another way. At 510, the two signals are compared, and time delay is found between them, for example by finding a delay time that maximizes their cross-correlation. The time delay provides information about the diameter of the smaller vessels.

Although the dependence of the time delay on the smaller vessel cross section has been empirically confirmed, as will be described below in the “Examples” section, and the invention does not depend on any particular model explaining the reasons for the time delay, a calculation will be presented here, providing a possible explanation for the relation between the time delay and the smaller vessel cross section, based on a simplified model for the cardiac pressure wave in an arteriole.

A fluid of viscosity η flowing through a straight cylindrical tube of length L, and circular cross section with radius R, with a pressure difference P between the beginning and end of tube, will have a flow rate Φ given by πPR⁴/8 ηL, according to Poiseuille's law. The flow rate is the cross sectional area πR² times the average flow velocity V, so the average flow velocity V is given by PR²/8 ηL. If the walls of the tube are sufficiently elastic so that the cross-sectional area of the tube expands by about a factor of 2 for the difference between the maximum and minimum of a pressure wave, and the fluid is incompressible, then the pressure wave can only travel through the tube at about the average flow velocity of the fluid. This is generally true in arterioles, which have very elastic walls. Then the time delay for the pressure wave to travel the length of the tube is on the order of L/V, or 8 ηL²/PR². If, during vasoconstriction, the elasticity of the walls scales with radius R in such a way that the pressure wave always causes the cross sectional area to change by the same factor, then the time delay will scale like 1/R². For a typical arteriole of length L=1 mm and radius R=0.016 mm, a pressure drop along the arteriole of 6000 pascals (50 mm Hg, or about 40% of the systolic pressure at the heart), and taking the viscosity of blood as 4×10⁻³ pascal-seconds, we find a time delay of about 20 milliseconds. This is comparable to the observed time delays, in the absence of vasoconstriction, of about 30 to 40 milliseconds, observed in the test described below in the “Examples” section, and better agreement should not be expected given the approximations made in deriving this expression for time delay.

At 512, the time delay is optionally compared to a time delay found at other times or in other parts of the body, optionally in the same way as this time delay. At 514, conclusions are drawn about the cross section of the small blood vessels. These conclusions need not involve absolute measures of the cross section dimensions, but could involve only changes in the cross section over time, possibly only about the direction of change. Additionally or alternatively, the conclusions could involve differences in the cross section, possibly only the sign of the difference, between this part of the body and other parts of the body.

In some embodiments of the invention, conclusions are drawn about the cross section of small blood vessels, based on whether or not the time delay is greater than a threshold value. For example, the threshold value is between 40 and 70 milliseconds, and if the time delay exceeds the threshold value, then conclusions are drawn that small blood vessels being measured exhibit vasoconstriction. Optionally, the threshold is specific for a patient, and/or for a particular method of measurement. Optionally, the threshold is determined by earlier testing of that patient, and is stored in a controller, such as controllers 118, 218, 318 or 418 of FIGS. 1-4 respectively, that performs the step of drawing conclusions about the cross section of the small blood vessels at 514.

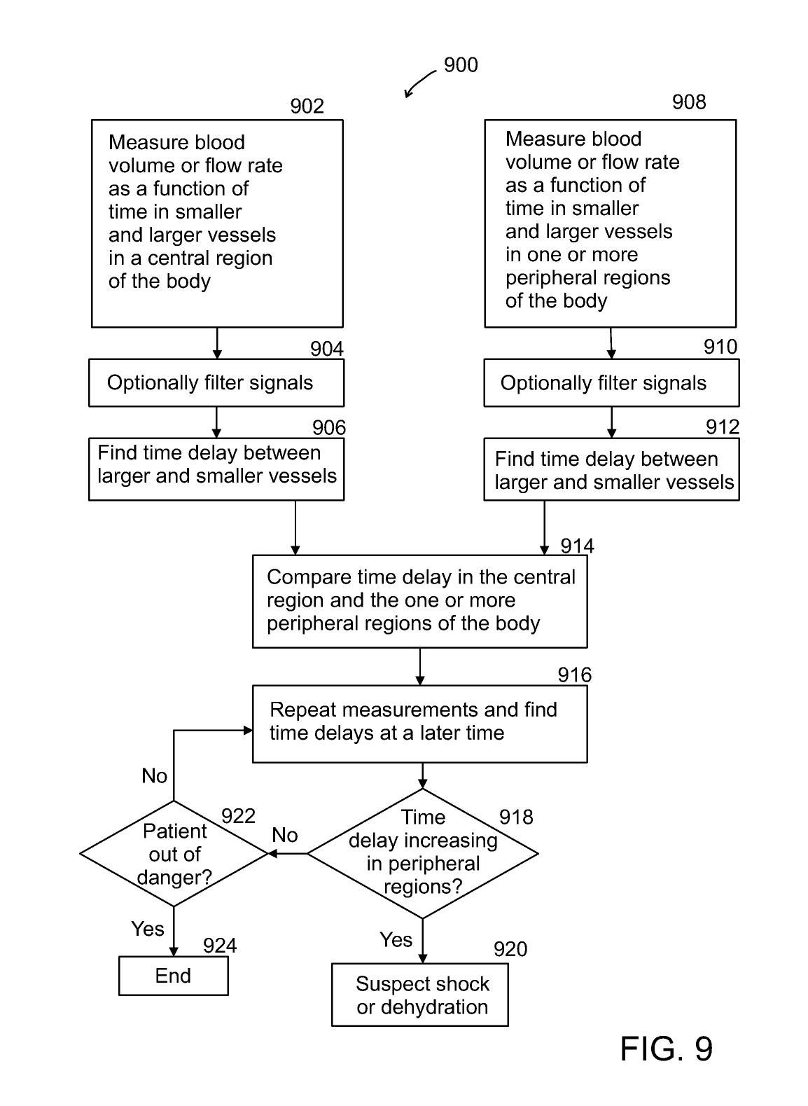

In general, a larger time delay means smaller cross section of the smaller blood vessels, at least if the smaller blood vessel walls are not also becoming more rigid when the diameter gets smaller. That seems to be the case with normal, reversible vasoconstriction in healthy subjects, as indicated by the observations described below under “Examples.” That data was obtained by inducing vasoconstriction by cooling part of the body. But vasoconstriction can also be sign of such dangerous medical conditions as shock and dehydration, and the method of flowchart 500 can be used to help diagnose such conditions, as will be described in more detail in the description of FIG. 9. In those cases, vasoconstriction occurs first in peripheral parts of the body, and can work its way closer to the central parts of the body, i.e. closer to the trunk, as the condition gets more severe. Monitoring vasoconstriction in such circumstances can be clinically useful, and it is not necessary to be able to calibrate the time delay to the exact diameter of the smaller blood vessels. It may be enough to observe qualitatively that the small blood vessel cross section is decreasing in time, more severely in peripheral parts of the body.

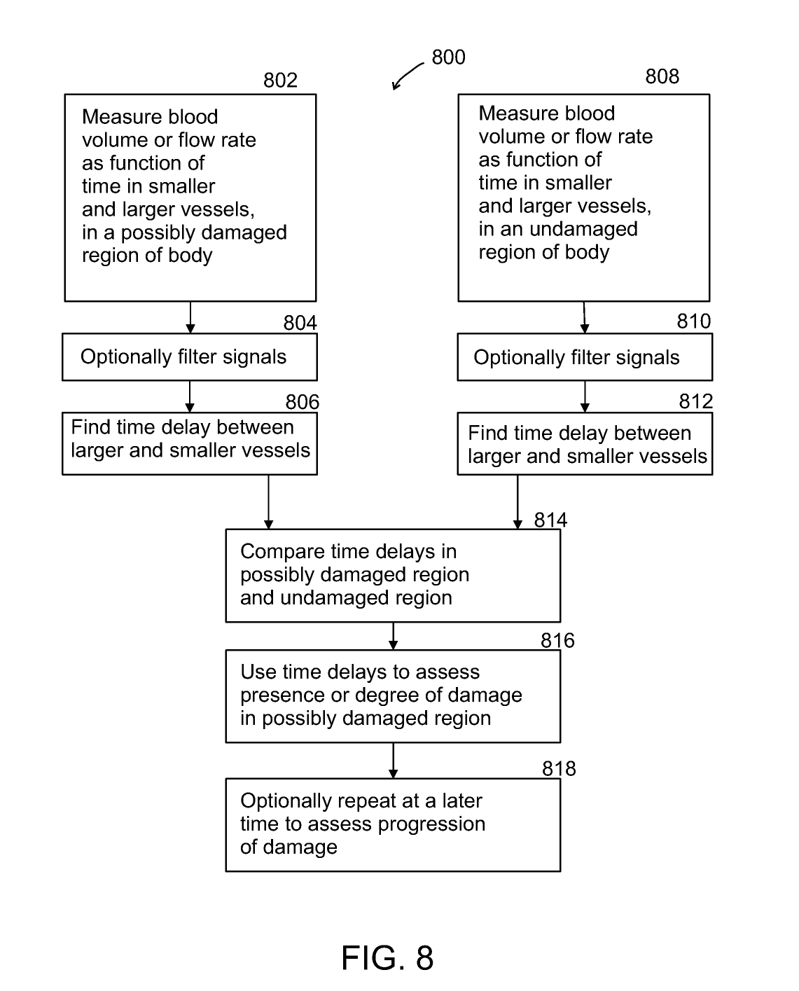

The method of flowchart 500 can also be used to measure changes in the cross sections of small blood vessels due to causes other than vasoconstriction. For example, pathologies such as diabetes, and atherosclerosis, may cause a narrowing of smaller arteries, and it can be clinically useful to monitor such changes over time, for example over months or years. In these cases, the relation between time delay and smaller artery cross section may not be so clear, because the artery walls may become stiffer, at the same time as the artery inner section becomes narrower. Increased stiffness of the artery wall by itself, with no change in inner diameter or cross sectional shape of the lumen, is expected to lead to a decrease in time delay, while decreasing inner diameter, for no change in stiffness or cross sectional shape of the lumen, is expected to lead to an increase in time delay. The time delay may also depend on the shape of the cross section of the lumen, for a given cross sectional area. In general, it may not be known, from first principles, whether progression of diabetes or atherosclerosis would be expected to lead to an increase or decrease in time delay in smaller arteries, and it may not even be the same for all patients. Even in these circumstances, measuring time delay repeatedly at different times can be useful, just by showing a change in time delay, in either direction. Also, particularly in the case of diabetic patients, there may be parts of the body where it is clear, from clinical indications, that small arteries have not yet been affect adversely by the disease, and these parts of the body may provide a reference for comparison, that can be used to evaluate the direction of change in time delay in smaller arteries in areas that are affected. Further details on using this method to evaluate patients with pathologies such as diabetes, are provided in the description of FIG. 8, below.

In some embodiments of the invention, if there is independent information about the cross section or expected cross section of smaller blood vessels, the time delay is used instead to estimate the pressure drop P along the blood vessels, which can be found from time delay and the cross section, for example using the equations given above, that relate the blood vessel radius R to the time delay τ and the pressure drop P along the blood vessel. The pressure drop P along the blood vessels can be used, in turn to estimate the mean arterial pressure, because they are typically in a fixed ratio. For example, the pressure drop along the arterioles is typically about 40% of the mean arterial pressure.

FIG. 6 shows a flowchart 600, describing another method of using measurements of the blood volume or flow rate as a function of time, at least for smaller blood vessels that branch off the larger vessels, to find information about the cross section of the smaller vessels. While the method of flowchart 500 uses a time delay between the signals from larger and smaller blood vessels for this purpose, the method of flowchart 600 uses a quantity representing the rise time or rate of rise of the blood volume or flow rate, at least for the smaller blood vessels, to find information about their cross section. Any other measurement that provides the form of a pressure wave as a function of time, such as oxygen or carbon dioxide level in blood or tissue, can also be used, instead of blood volume or flow rate.

At 602, the blood volume or flow rate is measured in the smaller blood vessels, as a function of time, using for example any of the methods shown in FIGS. 1-4. At 604, a signal of this measurement is optionally low-pass filtered, to remove high frequency noise, but not so strongly filtered as to seriously distort the shape of the pressure wave, and in particular the rise time and rate of rise. The signal is optionally detrended as well. At 606, the filtered signal is used to find a quantity representing the rise time or rate of rise of the signal during the interval when the pressure is rising in the blood vessel, for the smaller blood vessels. Such a quantity may be defined in a number of different ways. The total rise time may be used, defined as the time from the minimum to the maximum of the signal when it is rising. It is assumed, for purposes of defining the rise time, that the signal is defined to be more positive when the blood pressure is higher, so that the interval when the signal is rising is the same as the interval when the blood pressure is rising. Optionally, the rise time is normalized to the cardiac period, in the case of a cardiac wave. The average rate of rise may also be used, optionally normalized to the total rise, which is just the inverse of the rise time. Normalizing the rate of rise to the total rise, or some measure of the rise, has the potential advantage that the result does not depend on the gain of the signal, or the sensitivity of sensors used in the measurement, which may not always be well known, or consistent at different times and for different subjects.

Alternatively, the maximum rate of rise may be used, optionally normalized to the total rise, or to the rise up to the time of maximum rate of rise. The rise time from the minimum of the signal to the time of maximum rate of rise may also be used, optionally normalized to the cardiac period. The quantity may be a measure of a time asymmetry in the signal, for example a ratio of the rise time, or maximum or average rate of rise, with the falling time, or maximum or average rate of fall, or a difference between them, or a ratio of a Fourier component of the signal at the heart beat frequency to a Fourier component of the signal at twice the heart beat frequency, or a difference between them, or a ratio of Fourier components at odd integer multiples of the heart beat frequency to the Fourier components at even integer multiples of the heart beat frequency, or a difference between them.

Optionally, a similar quantity is found for the larger blood vessel or vessels, at 608, using any of the methods described in FIGS. 1-4. Alternatively, a different method is used to find a quantity representing rise time or rate of rise for larger blood vessels. For example, a mechanical blood pressure sensor, outside or inside the body, may be used to measure the blood pressure as a function of time in a larger artery, and a rise time or rate of rise may be found from that data. If a quantity representing rise time or rate of rise is found for larger blood vessels, using any method, then the quantities representing the rise time or rate of rise are optionally compared, for the larger and smaller blood vessels, at 610.

At 612, the quantity representing the rise time or rate of rise is optionally compared to its value found at other times, or other parts of the body.

At 614, conclusions are drawn, from this quantity and/or its changes in time and/or differences from place to place, about the cross section of small blood vessels, and/or their changes over time, and/or how they differ in different parts of the body. In general, having a longer rise time, or a slower rate of rise, is an indication of smaller cross section of the smaller blood vessels, similar to having a longer time delay in the method of flowchart 500. The rise time, or rate of rise, can be used in ways similar to the way the time delay can be used, as described above for FIG. 5. Like the time delay, the rise time and rate of rise may be affected in an opposite way, by increases in stiffness of the artery walls, to how they are affected by narrowing of the lumen of the blood vessel.

As in method 500, if there is independent information about the cross section or expected cross section of the smaller blood vessels, then the quantity representing the rise time or rate of rise is optionally used to estimate the pressure drop along the blood vessels, and the mean arterial pressure.

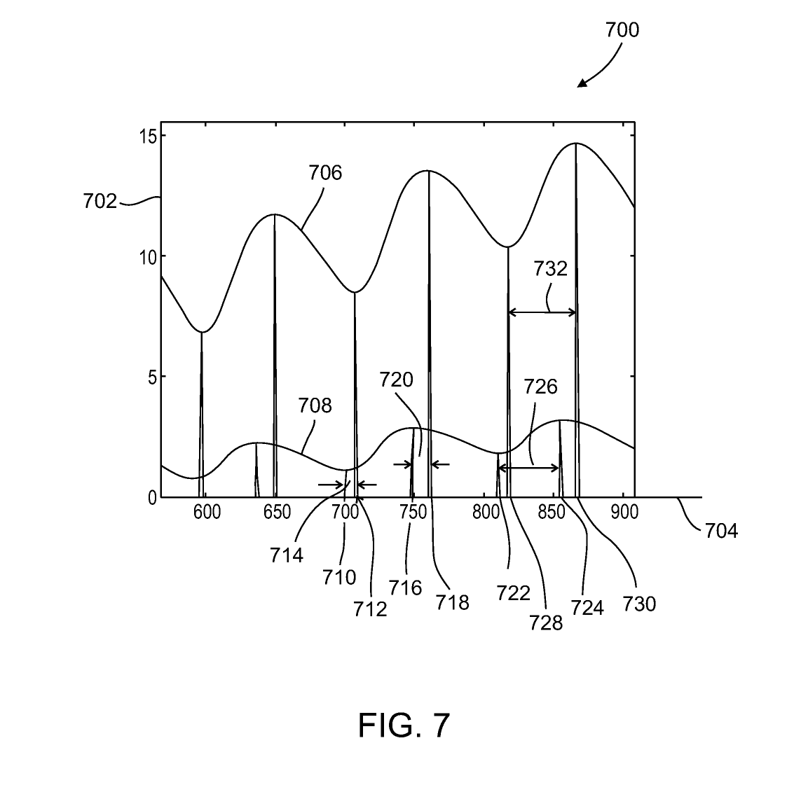

FIG. 7 shows a plot 700 of photoplethysmographic (PPG) signals for green and near infrared light, for the same location on the body of a test subject, to illustrate how the time delay and rise time may be found from the signals. The signals were obtained with a PPG system similar to system 100 shown in FIG. 1. The amplitude of the signal is plotted on a vertical axis 702, in arbitrary units, and the time is shown on a horizontal axis 704, also in arbitrary units. Curve 706 is the PPG signal using green light, which is sensitive primarily to the blood volume in the arterioles, while curve 708 is the PPG signal using near infrared light, which is sensitive primarily to the blood volume in the artery or arteries that the arterioles are branching off from. The signals have been low-pass filtered to remove noise, but still show the general shape of pressure waves in the artery and the arterioles. The signals have been inverted so that a more positive value of the signal indicates a greater volume of blood, even though a greater volume of blood results in a lower intensity of light scattered from the tissue, since the green light used for signal 706 and the near infrared light used for signal 708 are both absorbed more by blood than the surrounding tissue. Optionally, signals 706 and 708 are de-trended before finding the time delay or rise time, to remove drift in the signal from one cardiac period to the next that can distort the shape of the signal, although that was not done with signals 706 and 708 shown in FIG. 7.

To find a time delay between signal 706 and signal 708, a time difference is found for corresponding points on curve 706 and curve 708. For example, minima of the two signals, for the same cardiac cycle, may be used to find the time delay. Time 710 is a minimum of curve 708, and time 712 is the minimum of curve 706 for the same cardiac cycle. A difference 714 between time 712 and time 710 is optionally used as the time delay for these two signals. Alternatively, maxima of the two signals, for the same cardiac cycle, may be used to find the time delay. Time 716 is a maximum of curve 708, and time 718 is a maximum of curve 706 for the same cardiac cycle. A difference 720 between time 718 and time 716 is optionally used as the time delay between these two signals. Although time delay 714 is different from time delay 720, due to the different shape of curves 706 and 708, the time delay may be meaningfully compared at different times, and/or at different parts of the body, if the time delay is defined consistently. Still other procedures for measuring time delay include looking at the time difference of an inflection point, for example the time of greatest rate of rise, or the time of greatest rate of fall, for the two signals, or looking at the time difference between points that are half-way between the local minimum and local maximum in amplitude, or in time, for the two signals. The time delay can also be found by finding a time delay that maximizes a cross-correlation between the two signals. Optionally, the time delay, however it is found, is averaged over multiple cardiac periods, for example to reduce noise.

FIG. 7 also illustrates how the rise time may be found from the signals, either signal 706 which primarily measures the blood volume of arterioles, or signal 708 which primarily measures the blood volume of an artery that the arterioles are branching off from. The rise time may be defined, for example, as the time from the minimum of a cardiac cycle to the maximum of the same cycle. Time 722 is a minimum of curve 708, and time 724 is the maximum of curve 708 for the same cardiac cycle. The difference 726 between times 722 and 724 is the rise time of curve 708, for that cardiac cycle. Time 728 is a minimum of curve 706, and time 730 is the maximum of curve 706 for the same cardiac cycle. The difference 732 between times 728 and 730 is the rise time of curve 706 for that cardiac cycle. Rise time 732 is longer than rise time 726, and this difference is caused by the viscous drag of the blood in the arterioles, which prevents the blood in the arterioles from flowing as quickly during the rapid rise in arterial blood pressure, as it does in the larger arteries. This difference in rise time is used, in some embodiments of the invention, to estimate the cross section of the arterioles, and/or to compare the cross section of the arterioles in different times and/or places.

Optionally, before finding the time delay, or the rise time, the signal is examined to make sure that it is a good signal. For example, if the signal comes from arteries, it is examined to verify that its dominant component is at a reasonable cardiac frequency, optionally between 0.5 and 2 Hz.

FIG. 8 is a flowchart for a method of assessing or monitoring damage to small blood vessels due a pathological condition such as diabetes, using the method of FIG. 5. At 802, a quantity that serves as an indication of a pressure wave in blood vessels, such as blood volume or blood flow rate, is measured in a larger branching blood vessel and in the smaller blood vessels that branch off from it, for example using one of the systems shown in FIGS. 1-4, in a part of a body of a patient that is believed to have damage from a disease, such as diabetes, that can damage small blood vessels. Signals from these measurements are optionally low-pass filtered, at 804, and optionally detrended. A time delay between the two signals, for the larger and smaller blood vessels, is found at 806. At 808, measurements are made, similar to the measurements made at 802, but for a part of the body where the small blood vessels are believed to be undamaged, or less damaged, by the pathological condition. The signals from these measurements are optionally filtered at 810, and a time delay between the larger and smaller blood vessels is found at 812.

At 814, the time delays from the region believed to be damaged, and the region believed to be undamaged or less damaged, are compared, and results of the comparison are used, at 816, to assess the presence or degree of damage to small blood vessels, in the region believed to be damaged. In the case of diabetes, the time delay in the damaged region may be greater than or less than the time delay in the undamaged region, depending on whether increased viscous drag from narrowing of the blood vessels has a greater effect or smaller effect on the time delay than increased stiffness of the blood vessel walls. In either case, once the sign of the change in time delay, associated with the pathological condition in this patient, is known, at least for the part of the body examined, future measurements, at least in the same part of the body, can optionally be used, at 818, to judge whether the pathological condition has progressed further.

Optionally, if the method is being used to monitor a patient with diabetes, which often effects small blood vessels only in scattered localized regions of tissue without affecting other regions as much or at all, then an array of sets of sensors and detectors, each set similar to those shown in FIG. 1, or 2, or an array of sets of electrode units similar to those shown in FIG. 3, is used over a large area on a general part of the body, for example the foot, that is likely to be affected in some locations, in order to monitor the whole area at once.

The method of flowchart 800 may be particular suited for assessing damage to small blood vessels due to diabetes, since diabetes typically causes such damage only in some parts of the body and not in others, so it is usually possible to find regions, known to be relatively undamaged by diabetes, which can be used as a reference. The method of flowchart 800 may be less suited for assessing damage to small blood vessels due to atherosclerosis, since such damage may be more widespread throughout the body, and it may be difficult to find undamaged areas for comparison, but it may still be possible to use the method of flowchart 800 for assessing damage to small blood vessels due to atherosclerosis.

In some embodiments of the invention, damage to small blood vessels, due to a pathological condition, is assessed using a method similar to that of flowchart 800, but instead of finding time delays in damaged and undamaged regions and comparing them, using the method of FIG. 5, a quantity representing rise time or rate of rise is found and compared for damaged and undamaged regions, using the method of FIG. 6. Alternatively, both time delays and a quantity representing rise time or rate of rise are found and compared, which has the potential advantage of being more accurate because it is based on analyzing the data in more than one way.

FIG. 9 shows a flowchart 900, for a method of assessing shock or dehydration in a patient, from their vasoconstrictive effect, using the method of FIG. 5. Shock can be an indication of hidden internal bleeding in a trauma patient, and having a way to detect it early or to continuously monitor for it in a non-invasive way, using inexpensive equipment that could be carried in an ambulance or used routinely in an emergency room, could potentially save lives. The method of flowchart 900 uses the fact that, in shock or in dehydration, peripheral blood vessels tend to undergo vasoconstriction first, in order to preserve the volume of blood in the central region of the body, and the area of vasoconstriction increases, towards the center of the body, the trunk, if shock or dehydration persists. Using the method of FIG. 5 to detect a trend in vasoconstriction, in time and in different parts of the body, may be easier than using the method of FIG. 5 to assess a degree of vasoconstriction absolutely, at only one time and one part of the body.

At 902, a quantity that serves as an indication of a pressure wave in blood vessels, such as blood volume or blood flow rate, is measured in a larger branching blood vessel and in the smaller blood vessels that branch off from it, for example using one of the systems shown in FIGS. 1-4, in a part of a central part of the body of a patient. Signals from these measurements are optionally low-pass filtered, at 904, and optionally detrended. A time delay between the two signals, for the larger and smaller blood vessels, is found at 906. At 908, measurements are made, similar to the measurements made at 902, but for one or more peripheral parts of the body. The signals from these measurements are optionally filtered at 910, and a time delay between the larger and smaller blood vessels is found at 912. Optionally, similar measurements are made and time delays are found for several different peripheral parts of the body that are at increasing distances from the central part of the body, in order to determine whether vasoconstriction increases with distance from the central part of the body, as would be expected in a patient exhibiting shock of dehydration. Measurements at multiple locations can also be made to reduce error.

At 914, the time delays are compared in the central part of the body and in the one or more peripheral parts. Optionally, an estimation is made from these measurements at a single time as to whether the patient is exhibiting increasing vasoconstriction going further out from the central part of the body. At 916, the measurements are repeated, and the time delays found, at a later time. If, at 918, it is found that the time delay is increasing with time, indicating increased vasoconstriction, in peripheral regions of the body more than in the central part of the body, and especially if this trend is strongest in the most peripheral regions, this is an indication that the patient may be suffering from shock or dehydration, which are diagnosed, at least tentatively, at 920. Optionally, if the patient is being monitored for these conditions, then medical personnel are alerted at this time, for example through a cell phone or Bluetooth device, or by sounding an alarm in a room where the patient is located. If no such trend of increasing vasoconstriction in peripheral parts of the body is found, and if patient is judged to be out of danger at 922, then the procedure is ended at 924. If the patient is not judged to be out of danger, then measurements continue to be made, and time delays found, at later times, at 916.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

A test was made, using a PPG system similar to that described in FIG. 1, and the method of FIG. 5, on the forearm of subjects, before and after immersing the arm in cold water. The measurements were performed according to the following protocol:

1. The time delay was measured between green and near infrared PPG signals, on the forearm of the subject closer to the wrist, for 20 seconds, before immersing the subject's arm in cold water.

2. The subject's forearm was immersed in cold water, and the surface temperature of the forearm was measured once a minute with a non-contact IR sensor approved by the FDA for measuring body temperature, until it had fallen to 22 degrees C.

3. The area of the forearm to be measured was quickly dried, and the time delay was measured again for 20 seconds.

4. The surface temperature of the forearm, the time delay, and the uncertainty in the time delay, were recorded before and after cooling, and the room temperature was recorded.

5. The procedure was repeated 8 times at intervals of at least 24 hours.

The results of the experiment are shown below in Table 1. The time delay before the arm was cooled had a mean value of 34 milliseconds and a standard deviation of 14 milliseconds, with most of that standard deviation due to uncertainty in the measured value. After the arm was cooled, the time delay had a mean value 69 milliseconds, with a standard deviation of 24 milliseconds. A large part of that standard deviation was due to the last three values, which were significantly lower than the other values, probably because the room temperature was 20 or 21 degrees C. on those days, while on the other days it was 18 or 19 degrees. Apparently the higher room temperature resulted in significantly less vasoconstriction when the arm was cooled. If the last three values are excluded, then the mean value of the time delay after the arm was cooled was 83 milliseconds, with a standard deviation of 6 milliseconds, which by chance is even less than the uncertainty in the measured value. The mean and standard deviation for the time delay before cooling the arm are nearly unchanged if the last three values are excluded. The difference in time delay before and after cooling the arm is very statistically significant, and shows that the effect of narrowing the blood vessels, which would increase the time delay, is more important than any increase in stiffness of the blood vessel walls, which would tend to decrease the time delay. If the time delay is proportional to the inverse square of the blood vessel inner diameter, as expected from the simple calculation presented above in the description of FIG. 5, and there is no change in the stiffness of the blood vessel walls, then cooling the arm is decreasing the blood vessel inner diameter to 65% of its value before cooling, which seems like a reasonable value.

TABLE 1
Test data for time delay before and after cooling subject's arm

| | | Data before cooling | | | | Data after cooling | | | | | |
|---|---|---|---|---|---|---|---|---|---|---|---|
| | | | | Delay | | | | Delay | | | Room |
| Tw = 18° C. | | Temp., ° C. | Name of Data | Before, | STD | Temp., ° C. | Name of Data | After, | STD | Temp., ° C. | Temperature, |
| # | Time | Before | File | ms | ms | After | File | ms | ms | After - Right | C. |

| | | | | | | | | | | | |
|---|---|---|---|---|---|---|---|---|---|---|---|
| 1 | 06:35:00 PM | 34.9 | data1\before.txt | 56.9 | 18.6 | 24.0-25.6 | data1\after.txt | 80.53 | 12 | 25.6 | 19 |
| 2 | 08:14:00 PM | 35.2 | data2\before.txt | 38.8 | 12.7 | 23.5-25.5 | data2\after.txt | 91.11 | 20.65 | 25.5 | 18 |
| 3 | 06:15:00 PM | 35.1 | [symbol] ata3\before6R.t [symbol] | 26.84 | 10.2 | 23.8-25.1 | data3\after6R.txt | 84.56 | 9.4 | 25.1 | 18 |
| 4 | 03:25:00 PM | 34.9 | data4\before.txt | 32.14 | 11.7 | 23.2-24.2 | data4\after.txt | 83.45 | 10.8 | 24.2 | 18 |
| 5 | 05:22:00 PM | 35.6 | data5\before3.tx [symbol] | 34 | 8 | 24.5-25.1 | data5\after3.txt | 73.73 | 16.7 | 25.1 | 18 |
| 6 | 05:34:00 PM | 35.2 | data6\before.txt | 19.47 | 5.4 | 23.8-25.2 | data6\after.txt | 82.37 | 9.9 | 25.2 | 18 |
| 7 | 10:53:00 PM | 35.8 | data7\before.txt | 13.42 | 4.8 | 23.2-24.9 | data7\after3.txt | 15.33 | 2.5 | 24.9 | 21 |
| 8 | 04:01:00 PM | 35.4 | data8\before.txt | 35.96 | 9.8 | 23.7-24.6 | data8\after.txt | 50 | 12.8 | 24.6 | 20 |
| 9 | 11:41:00 PM | 35.2 | data9\before.txt | 51.94 | 13.3 | 22.2-23.5 | data9\after3.txt | 62.2 | 14.6 | 23.5 | 20 |

[symbol]

indicates data missing or illegible when filed

These test results show that the optical system of FIG. 1, using the method of FIG. 5, is capable of detecting reversible vasoconstriction of arterioles due to exposure to cold, with reasonable precision, and shows that the dominant effect is the increase in time delay due to the narrowing of the inner diameter of the blood vessel, rather than any decrease in time delay that might occur due to an increase in stiffness of the arteriole walls. This conclusion might not apply to long-term changes in arteries and arterioles due to pathological conditions such as diabetes and atherosclerosis, for which the increase in wall stiffness might be greater, and the cross-section of the lumen may change in shape as well as in size. But it seems likely that changes in the cross section of blood vessels in those cases can also be detected and monitored with this technique.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A system for measuring an indication of blood vessel cross section in a subject, the system comprising:
a) a sensor adapted to perform measurements indicative of a pressure wave in blood vessels in the subject, and to generate first and second signals of the measurements, wherein a larger blood vessel contributes more, relative to smaller blood vessels branching off it, for the first signal than for the second signal; and
b) a signal processor adapted to find a time delay between the first and second signals, and to use the time delay to find the indication of cross section for the smaller blood vessels.

2. A system according to claim 1, wherein the sensor is adapted to perform the measurements when it is placed adjacent to a surface of the subject's body, with the pressure wave contributing to the first signal from deeper beneath the surface, on average, than the pressure wave contributes to the second signal.

3. A system according to claim 1 or claim 2, wherein the signal processor is adapted to find the time delay by finding a difference in timing in a minimum of the pressure wave for the first and second signals.

4. A system according to claim 1 or claim 2, wherein the signal processor is adapted to find the time delay by finding a difference in timing in a maximum rate of increase of the pressure wave for the first and second signals.

5. A system according to claim 1 or claim 2, wherein the signal processor is adapted to find the time delay by finding a difference in timing in a maximum of the pressure wave for the first and second signals.

6. A system according to any of the preceding claims, adapted to perform the measurements non-invasively.

7. A system according to any of claims 1-5, adapted to perform the measurements inside the body, or when a internal part of the body is exposed during surgery.

8. A system according to any of the preceding claims, wherein the measurements indicative of a pressure wave comprise measurements of blood volume in the larger blood vessel and in the smaller blood vessels branching off from it.

9. A system according to any of the preceding claims, wherein the measurements indicative of a pressure wave comprise measurements of flow rate in the larger blood vessel and in the smaller blood vessels branching off from it.

10. A system according to any of the preceding claims, wherein the measurements indicative of a pressure wave comprise optical measurements.

11. A system according to claim 10, wherein the sensor is adapted to perform the measurements when it is placed adjacent to a surface of the subject's body, and the first signal is a signal of an optical measurement using a first set of wavelengths of light, while the second signal is a signal of an optical measurement using a second set of wavelengths of light that do not penetrate as far beneath the surface of the body as the first set of wavelengths of light.

12. A system according to claim 10 or claim 11, wherein the sensor comprises:
a) a first light source and first detector adapted to be placed a first distance apart on a surface of the subject's body, light from the first light source scattering from beneath the surface to the first detector to generate the first signal; and
b) a second light source and a second light detector, one or both of them different respectively from the first light source and the first light detector, adapted to be placed a second distance apart, smaller than the first distance, light from the second light source scattering from beneath the surface to the second detector to generate the second signal.

13. A system according to any of claims 10-12, wherein the measurements comprise one or more of measurements of oxygen level and measurements of carbon dioxide level, in the blood or tissue or both.

14. A system according to claim 1, wherein the measurements indicative of a pressure wave comprise ultrasound measurements.

15. A system according to claim 14, wherein the sensor is adapted to perform the measurements when it is placed adjacent to a surface of the subject's body, and the first signal is a signal of an ultrasound measurement using a first set of frequencies of ultrasound, while the second signal is a signal of an ultrasound measurement using a second set of frequencies of ultrasound that do not penetrate as far beneath the surface of the body as the first set of wavelengths of light.

16. A system according to claim 14 or claim 15, wherein the sensor comprises:
a) a first ultrasound transducer and first receiver adapted to be placed a first distance apart on a surface of the subject's body, ultrasound from the first ultrasound transducer scattering from beneath the surface to the first receiver to generate the first signal; and
b) a second ultrasound transducer, the same as or different from the first ultrasound transducer, and a second receiver, the same as or different from the first receiver, adapted to be placed a second distance apart on the surface of the subject's body, smaller than the first distance, ultrasound from the second ultrasound transducer scattering from beneath the surface to the second receiver to generate the second signal.

17. A system according to claim 1, wherein the measurements indicative of a pressure wave comprise electrical impedance measurements.

18. A system according to claim 17, wherein the sensor comprises:
a) a first pair of electrode units, adapted to be placed on a surface of the subject's body a first distance apart, and to measure a first impedance between them to generate the first signal; and
b) a second pair of electrode units, one or both of the units in the second pair differing from the electrode units in the first pair, adapted to be placed on a surface of the subject's body a second distance apart, shorter than the first distance, and to measure a second impedance between them to generate the second signal.

19. A system according to any of the preceding claims, wherein the sensor has a time resolution better than 20 milliseconds.

20. A system according to any of the preceding claims, wherein the signal processor is adapted to use the time delay by comparing the time delay to a threshold value for that subject, stored in a memory of the signal processor, and to find the indication that the smaller blood vessels are undergoing vasoconstriction if the time delay is greater than the threshold value.

21. A system for measuring an indication of blood vessel cross section in a subject, the system comprising:
a) a sensor adapted to perform at least a first set of measurements indicative of a pressure wave in blood vessels in the subject, and to generate at least a first signal of the first set of measurements; and
b) a signal processor adapted to find a first quantity indicative of rise time or rise rate of the pressure wave from the first signal, and to use the first quantity to find the indication of cross section for the blood vessels.

22. A system according to claim 21, wherein the sensor is adapted to perform a second set of measurements indicative of a pressure wave in blood vessels in the subject and to generate a second signal, wherein a larger blood vessel contributes more, relative to smaller blood vessels branching off it, for the first signal than for the second signal, and wherein the signal processor is adapted to find a second quantity indicative of rise time or rise rate of the pressure wave from the second signal, and to use a comparison of the first quantity to the second quantity, to find the indication of cross-section for the blood vessels.

23. A system according to claim 21 or claim 22, also including a blood pressure sensor that measures blood pressure as a function of time for a larger blood vessel than the blood vessels used for the first set of measurements, wherein the signal processor is adapted to find a second quantity indicative of rise time or rate of rise of the blood pressure of the larger blood vessel, and to use a comparison of the first quantity to the second quantity, to find the indication of cross-section for the blood vessels.

24. A system according to any of claims 21-23, wherein the sensor comprises an optical sensor.

25. A system according to any of claims 21-24, wherein the sensor comprises an ultrasound sensor.

26. A system according to any of claims 21-25, wherein the sensor comprises an electrical impedance sensor.

27. A system according to any of claims 21-26, wherein the sensor has a time resolution better than 20 milliseconds.

28. A method for measuring an indication of blood vessel cross section in a subject, the system comprising:
a) making or providing a first set of measurements indicative of a pressure wave in a larger blood vessel and in smaller blood vessels that branch off it, and generating a first signal from the first set of measurements;
b) making or providing a second set of measurements indicative of the pressure wave in the larger blood vessel and in the smaller blood vessels that branch off it, and generating a second signal from the second set of measurements, the larger blood vessel contributing more, relative to the smaller blood vessels, for the first signal, than it does for the second signal;
c) finding time delay between the first signal and the second signal; and
d) using the time delay to find the indication of blood vessel cross section for the smaller blood vessels.

29. A method according to claim 28, wherein the larger blood vessel contributes more than the smaller blood vessels to the first signal, and the smaller blood vessels contribute more than the larger blood vessel to the second signal.

30. A method according to claim 28 or claim 29, wherein the measurements are made on a surface of the subject's body, the first set of measurements respond to the pressure wave with a fall-off down to a first characteristic depth, and the second set of measurements respond to the pressure wave with a fall-off down to a second characteristic depth, less than the first characteristic depth.

31. A method according to claim 30, wherein the first characteristic depth is at least 2 times as great as the second characteristic depth.

32. A method according to claim 30, wherein the first characteristic depth is greater than 5 mm.

33. A method according to any of claims 30-32, wherein the second characteristic depth is less than 5 mm.

34. A method according to any of claims 28-33, comprising making the first and second sets of measurements again at another time, finding the time delay at the other time, and using the time delays at both times to find the indication of blood vessel cross section.

35. A method according to claim 34, wherein the indication of blood vessel cross section comprises a change in blood vessel cross section or a direction of a change in blood vessel cross section over time.

36. A method according to any of claims 28-35, comprising making the first and second sets of measurements again at another place on the subject's body, finding the time delay at the other place, and using the time delays at both places to find the indication of blood vessel cross section.

37. A method according to claim 36, wherein the indication of blood vessel cross section comprises a difference in blood vessel cross section, or a direction of difference in blood vessel cross section, between the two places.

38. A method according to any of claims 28-37, also including using the time delay, and separately obtained information about a cross section or expected cross section of the smaller blood vessels, to estimate mean arterial pressure.

39. A method according to any of claims 28-38, wherein the indication of blood vessel cross section comprises one or more of a measure of vasoconstriction, change in vasoconstriction over time, and difference in vasoconstriction in different parts of the body.

40. A method according to claim 39, wherein the indication of blood vessel cross section comprises a difference in vasoconstriction between a peripheral and a central part of the body, and the method also includes diagnosing shock, dehydration, or both, from the difference in vasoconstriction.

41. A method according to any of claims 28-40, wherein the indication of blood vessel cross section comprises one or more of a measure of damage to small blood vessels due to a pathological condition, a change in damage to small blood vessels over time, due to a pathological condition, and a difference in damage to small blood vessels in different parts of the body.

42. A method according to claim 41, wherein the indication of blood vessel cross section comprises a difference in damage to small blood vessels in different parts of the body, and the method also includes assessing damage due to diabetes, from the difference in damage to small blood vessels between a part of the body damaged by diabetes, and an undamaged part of the body.

43. A method according to any of claims 28-42, wherein using the time delay to find the indication of blood vessel cross-section comprises finding an indication of vasoconstriction of arterioles if the time delay is longer than a critical value, and the critical value is between 40 and 70 milliseconds.

44. A method for measuring an indication of blood vessel cross section in a subject, the system comprising:
a) making or providing at least a first set of measurements indicative of a pressure wave in smaller blood vessels that branch off a larger blood vessel, and generating a first signal from the first set of measurements;
b) finding a quantity indicative of rise time or rate of rise of the pressure wave in the smaller blood vessels; and
c) using the quantity indicative of rise time or rate of rise to find the indication of blood vessel cross section for the smaller blood vessels.

45. A method according to claim 44, comprising making the set of measurements again at another time, finding the quantity indicative of rise time or rate of rise at the other time, and using the quantities at both times to find the indication of blood vessel cross section.

46. A method according to claim 45, wherein the indication of blood vessel cross section comprises a change in blood vessel cross section or a direction of a change in blood vessel cross section over time.

47. A method according to any of claims 44-46, comprising making the first set of measurements again at another place on the subject's body, finding the quantity indicative of rise rate or rate or rise at the other place, and using the quantities at both places to find the indication of blood vessel cross section.

48. A method according to claim 47, wherein the indication of blood vessel cross section comprises a difference in blood vessel cross section, or a direction of difference in blood vessel cross section, between the two places.

49. A method according to any of claims 44-48, also including using the quantity indicative of rise time or rate of rise, and independent information about a cross section or expected cross section of the smaller blood vessels, to estimate mean arterial pressure.

50. A method according to any of claims 44-49, wherein the indication of blood vessel cross section comprises one or more of a measure of vasoconstriction, change in vasoconstriction over time, and difference in vasoconstriction in different parts of the body.

51. A method according to claim 50, wherein the indication of blood vessel cross section comprises a difference in vasoconstriction between a peripheral and a central part of the body, and the method also includes diagnosing shock, dehydration, or both, from the difference in vasoconstriction.

52. A method according to any of claims 44-51, wherein the indication of blood vessel cross section comprises one or more of a measure of damage to small blood vessels due to a pathological condition, a change in damage to small blood vessels over time, due to a pathological condition, and a difference in damage to small blood vessels in different parts of the body.

53. A method according to claim 52, wherein the indication of blood vessel cross section comprises a difference in damage to small blood vessels in different parts of the body, and the method also includes assessing damage due to diabetes, from the difference in damage to small blood vessels between a part of the body damaged by diabetes, and an undamaged part of the body.

54. A method according to any of claims 44-53, wherein the small blood vessels are less than 1 mm in inside diameter.

55. A method according to claim 54, wherein the small blood vessels are arterioles.

56. Apparatus for collecting information about blood vessel cross-section near a surface of the body, comprising:
at least one transmitter and at least one receiver; and
circuitry configured to activate said at least one transmitter and said at least one receiver to collect radiation scattered off two volumes in a body portion, a first volume being configured to include a predominant amount of scattering from arterioles and a second volume being configured to include a predominant amount of scattering from arteries from which said arterioles split off.

57. Apparatus for processing information about blood vessel cross-section near a surface of the body, comprising:
a signal receiving section which receives two signals, a first signal being including a predominant contribution of scattering from arterioles and a second signal including a predominant contribution of scattering from arteries from which said arterioles split off; and
circuitry which processes said signals to detect a difference in time between pulse wave in the two signals.
